(19) United States
(12) Patent Application Publication
(10) Pub. No.: US 2008/0033552 A1
(43) Pub. Date: Feb. 7, 2008

(54) Sensor Device

(75) Inventors: Sang-ho Lee, Busan (KR); Ho-yeong Kang, Gyeonggi-do (KR); Jeang-sub Park, Seoul (KR); Seung-hwa Yoo, Gyeonggi-do (KR)

Correspondence Address: NATH & ASSOCIATES, 112 South West Street, Alexandria, VA, 22314, US

(73) Assignee: Canon Kasushiki Kaisha, Tokyo (JP)

(21) Appl. No.: 11/596,853

(22) PCT Filed: May 17, 2005
(86) PCT No.: PCT/KR05/01451

§ 371 Date: Nov. 17, 2006

(51) Int. Cl. A61B 17/70

(52) U.S. Cl. 623/17.11; 382/131

(57) ABSTRACT

Provided is an intervertebral implant which is fixedly placed between spinous processes of adjacent vertebrae to maintain a predetermined space between the spinous processes and to prevent a relative displacement between superior and inferior facets of adjacent vertebrae. The intervertebral implant includes a spacer having two opposing notches for receiving two adjacent spinous processes and a band for securing the two spinous processes and the spacer, the spacer comprising a through-hole bored through sides of the spacer to allow the band to pass therethrough and depressions curved inwardly from outsides of the spacer to facilitate fastening of the band passed through the through-hole, and the band binding the two spinous processes and the spacer in a figure 8 form while passing through the through-hole to secure the two spinous processes and the spacer.

### BACKGROUND OF THE INVENTION

1. Field of the Invention

The present invention relates to an intervertebral implant, and more particularly, to an intervertebral implant which is fixedly placed between spinous processes of adjacent vertebrae to maintain a predetermined space between the spinous processes and to prevent a relative displacement between adjacent superior and inferior facets.

2. Background

FIG. 1 is a lateral view of a typical human spinal column. Referring to FIG. 1, a plurality of spinous processes 3 are positioned at the back of the human body and a plurality of vertebral bodies 8 are positioned on the opposite side. A vertebral nerve 1 is located in a space between the spinous processes 3 and the vertebral bodies 8. Interspinous ligaments 7 and ligamentum flava 6 are positioned between the spinous processes 3. Supraspinous ligaments 5 and a skin 20 run along the posterior surfaces of the spinous processes 3.

As aging proceeds, the human spinal column undergoes a retrogressive change. As a result, the space between the spinous processes 3 decreases (as represented by a dotted line (A)), and the ligamentum flava 6 thicken while losing their resilience and protrude anteriorly (as represented by a dotted line (B)). Therefore, the spinous processes 3 or the ligamentum flava 6 compress the vertebral nerve 1 or the nerve processes (not shown) connected to the vertebral nerve 1, which is called “spinal stenosis.”

Medication, physical therapy, and surgery have been utilized in the treatment of spinal stenosis. Surgical treatment is done when spinal stenosis cannot be managed through non-surgical treatments. The most common type of surgery done to treat spinal stenosis is removing bones or tissues compressing the vertebral nerve, and a screw gauge is then inserted into the spine to overcome spinal instability due to bone or tissue removal. However, this type of surgery involves general anesthesia since a large volume of bone or tissue is removed. In addition, a lengthy surgery and recovery period is required. Thus, it may be difficult to apply the surgery to physically weak elderly persons. Furthermore, unsatisfactory surgical outcomes such as complications occur frequently and the surgery can be expensive. As a solution to these problems, Korean Patent Laid-Open Publication No. 2002-0068035 discloses an intervertebral implant inserted between two spinous processes.

### SUMMARY

According to an aspect of the present invention, there is provided an intervertebral implant including a spacer having two opposing notches receiving two adjacent spinous processes and a band securing the two spinous processes and the spacer, the spacer including a through-hole bored through sides of the spacer to allow the band to pass therethrough and depressions curved inwardly from outsides of the spacer to facilitate fastening of the band passed through the through-hole, and the band binding the two spinous processes and the spacer in a figure 8 while passing through the through-hole to secure the two spinous processes and the spacer.

According to another aspect of the present invention, there is provided an intervertebral implant including a spacer having two opposing notches and receiving two adjacent spinous processes, an elastic folding portion connecting the two opposing notches and producing an elastic restoring force to counter an external force generated from the two spinous processes, two through-holes formed respectively on the two opposing notches, and a band binding the spacer and the two spinous processes by passing through the two through-holes.

According to still another aspect of the present invention, there is provided an intervertebral implant including an upper body having a first notch, a lower body having a second notch opposite the first notch, a cylindrical receiver formed on a lower portion of the upper body, and an insertion member formed on an upper portion of the lower body and partially inserted into the cylindrical receiver and having a first angle portion formed near an insertion front part and a second angle portion formed near an insertion rear part, the first angle portion and the second angle portion having different slopes.

### BRIEF DESCRIPTION OF THE DRAWINGS

The features, nature, and advantages of the present invention will become more apparent from the detailed description set forth below when taken in conjunction with the drawings in which like reference characters identify corresponding items throughout and wherein:

FIG. 1 is a lateral view of a typical human spinal column.

FIG. 2 is a view illustrating a conventional intervertebral implant.

FIG. 3 is a perspective view of a spacer according to an embodiment of the present invention.

FIG. 4 is a front view of a spacer according to an embodiment of the present invention.

FIG. 5 is a right side view of a spacer according to an embodiment of the present invention.

FIG. 6 is a perspective view of a strap according to the present invention.

FIGS. 7 and 8 are views illustrating the insertion of a spacer into the human body.

FIG. 9 is a diagrammatic view of a spinal column showing a spacer inserted between adjacent spinous processes.

FIGS. 10 through 13 are sequential views illustrating the installing of a spacer with a strap according to a first embodiment of the present invention.

FIG. 14 is a view illustrating a spacer installed with a strap according to a second embodiment of the present invention.

FIGS. 15 and 16 are sequential views illustrating the installing of the spacer with the strap according to the second embodiment of the present invention.

FIG. 17 is a diagrammatic lateral view of a spinal column showing a spacer inserted between adjacent spinous processes according to an embodiment of the present invention.

FIG. 18 is a diagrammatic view illustrating a lever system in the human body.

FIG. 19 is a diagrammatic view of a human spinal column illustrating normal articulation of facet joints.

FIG. 20 is a diagrammatic view showing the state of a spinal column after a spacer is inserted therein according to a conventional technique.

FIG. 21 is a diagrammatic view showing the state of a spinal column after a spacer is inserted therein according to the present invention.

FIG. 22 is a graph illustrating the profile slope of a first notch of an intervertebral implant according to an embodiment of the present invention plotted in the x-y plane.

FIG. 23 is a histogram illustrating the distribution of lower surface slope measurements of the third spinous process.

FIG. 24 is a histogram illustrating the distribution of lower surface slope measurements of the fourth spinous process.

FIG. 25 is a flow diagram illustrating a method of manufacturing a first notch of an intervertebral implant according to an embodiment of the present invention.

FIG. 26 is a graph illustrating the lower surface slope of the third spinous process plotted in the x-y plane.

FIG. 27 is a graph illustrating the lower surface slope of the fourth spinous process plotted in the x-y plane.

FIG. 28 is a graph illustrating the mean lower surface slope of the third and fourth spinous processes plotted in the x-y plane.

FIG. 29 is a schematic block diagram illustrating a spinal image clustering system according to an embodiment of the present invention.

FIG. 30 is a flow diagram illustrating a spinal image clustering according to an embodiment of the present invention.

FIG. 31 is a detailed flow diagram of a preparing operation S210 of the spinal image clustering of FIG. 30.

FIG. 32 shows a spinal sectional image of 256 gray levels with a selected volume of interest (VOI).

FIGS. 33 and 34 are respectively a sectional image of 256 gray levels for a selected VOI and a binarized sectional image of the 256 gray-levels image by image binarization.

FIG. 35 is a detailed flow diagram of a clustering operation S220 of the spinal image clustering of FIG. 30.

FIG. 36 is a detailed flow diagram of a representative image matching operation S270 of the spinal image clustering of FIG. 30.

FIG. 37 is an exemplary view of a representative image matching operation according to an embodiment of the present invention.

FIGS. 38 through 40 are sectional views illustrating assignment of gray-level values to a case image and a region of interest (ROI) of a matching template according to an embodiment of the present invention.

FIG. 41 is a perspective view illustrating a variable space that can be scanned by a matching template in a case according to an embodiment of the present invention.

FIG. 42 is a perspective view illustrating a spacer according to another embodiment of the present invention.

FIG. 43 is a diagrammatic lateral view of a spinal column illustrating the placement of the spacer of FIG. 42 between adjacent spinous processes.

FIG. 44 is a sectional view of the spacer of FIG. 43 fitted with a band.

FIG. 45 is a perspective view illustrating a spacer according to still another embodiment of the present invention.

FIG. 46 is a left side view of the spacer of FIG. 45.

FIG. 47 is a diagrammatic lateral view of a spinal column illustrating the placement of the spacer of FIG. 45 between adjacent spinous processes.

### DETAILED DESCRIPTION OF THE INVENTION

The word “exemplary” is used herein to mean “serving as an example, instance, or illustration.” Any embodiment described herein as “exemplary” is not necessarily to be construed as preferred or advantageous over other embodiments.

Overview

Hereinafter, the exemplary embodiments of the invention will be explained with drawings. The present invention will now be described more fully with reference to the accompanying drawings, in which preferred embodiments of this invention are shown. Advantages and features of the present invention and methods of accomplishing the same may be understood more readily by reference to the following detailed description of preferred embodiments and the accompanying drawings. The present invention may, however, be embodied in many different forms and should not be construed as being limited to the embodiments set forth herein. Rather, these embodiments are provided so that this disclosure will be thorough and complete and will fully convey the concept of the invention to those skilled in the art, and the present invention will only be defined by the appended claims. Like reference numerals refer to like elements throughout the specification.

General Structure

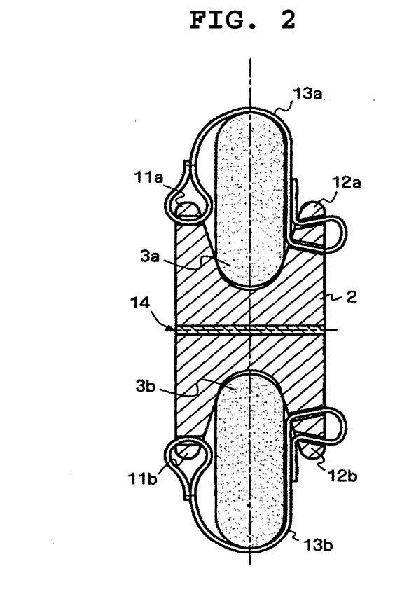

Referring to FIG. 2, an intervertebral implant includes a spacer 2 having two opposing notches suitable for receiving two spinous processes 3a and 3b of two vertebrae to be inserted between the two spinous processes 3a and 3b. Here, one of the two opposing notches is defined by two upper flanges 11a and 12a with inner walls and the other notch is defined by two lower flanges 11b and 12b with inner walls. The intervertebral implant also includes ties 13a and 13b for securing the spacer 2 to the two spinous processes 3a and 3b. These ties 13a and 13b surround surface portions of the spinous processes 3a and 3b.

After the intervertebral implant is inserted between the two spinous processes 3a and 3b, ends of the ties 13a and 13b are pulled to hold the ties 13a and 13b in position, resulting in securing of the spacer 2 to the two spinous processes 3a and 3b. In addition, to detect the position of the spacer 2 inserted between the spinous processes 3a and 3b, a radioopaque transversal member may be inserted into the spacer 2. The transversal member is sufficiently thin so that x-ray observation is not disturbed, and it is encased in a central housing 14.

Structural Implementation

An intervertebral implant according to the present invention includes a spacer maintaining a predetermined space between two adjacent spinous processes, and a strap binding the spacer and the two spinous processes.

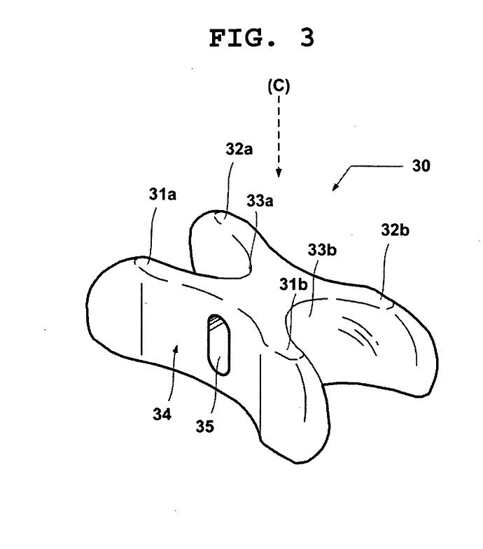

FIG. 3 is a perspective view of a spacer 30 according to an embodiment of the present invention. Referring to FIG. 3, the spacer 30 includes a first notch 33a, a second notch 33b, first flanges 31a and 32a, second flanges 31b and 32b, a first depression 34, a second depression located opposite the first depression (not shown), and a through-hole 35.

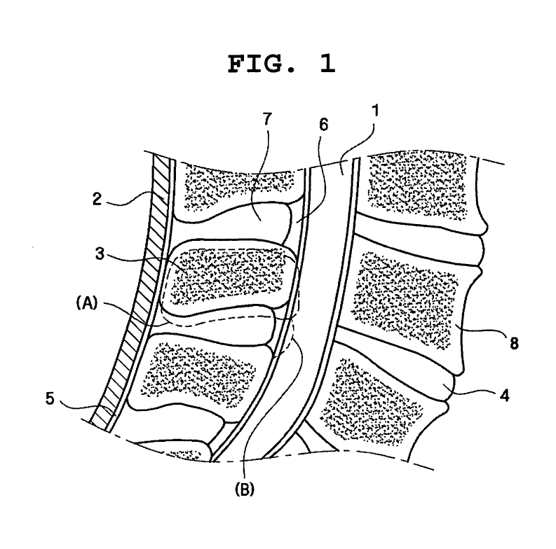

FIG. 4 shows the spacer 30 of FIG. 1 as viewed from the top. As shown in FIG. 4, the spacer 30 is symmetric about the top-bottom axis, but it may not be symmetric about the left-right axis.

The spacer 30 is inserted between two adjacent spinous processes 3a and 3b affected by spinal stenosis. The spacer 30 has the first notch 33a for receiving a lower portion of an upper spinous process among the two spinous processes, and the second notch 33b for receiving an upper portion of the other spinous process, i.e., a lower spinous process. The first and second notches 33a and 33b face opposite directions, and support an upward- and downward-directed compression force of the higher and lower spinous processes.

Generally, upper and lower portions of spinous processes have different shapes. That is, the lower portions of spinous processes are relatively narrow and elongated, whereas the upper portions are relatively wide and shortened. Thus, it is preferable that the dimensions of the first notch 33a and the first flanges 31a and 32a are different from those of the second notch 33b and the second flanges 31b and 32b so that the first notch 33a and the first flanges 31a and 32a are fitted with the lower portion of the upper spinous process, and the second notch 33b and the second flanges 31b and 32b are fitted with the upper portion of the lower spinous process.

The first notch 33a is defined by the first flanges 31a and 32a, which prevent left- and right-directed displacements of the upper spinous process, and the second notch 33b is defined by the second flanges 31b and 32b, which prevent left- and right-directed displacements of the lower spinous process.

The first depression 34 is formed on the left side of the spacer 30 and the second depression 36 is formed on the right side of the spacer 30. The first and second depressions 34 and 36 facilitate the pulling of both ends of a strap passed through the through-hole 35. In order to install the spacer 30 between adjacent spinous processes, the first depression 34 is curved inwardly from left outer edges of the first flange 31a and the second flange 31b, and the second depression 36 is curved inwardly from right outer edges of the first flange 32a and the second flange 32b. The inward curvature can be changed according to user requirements.

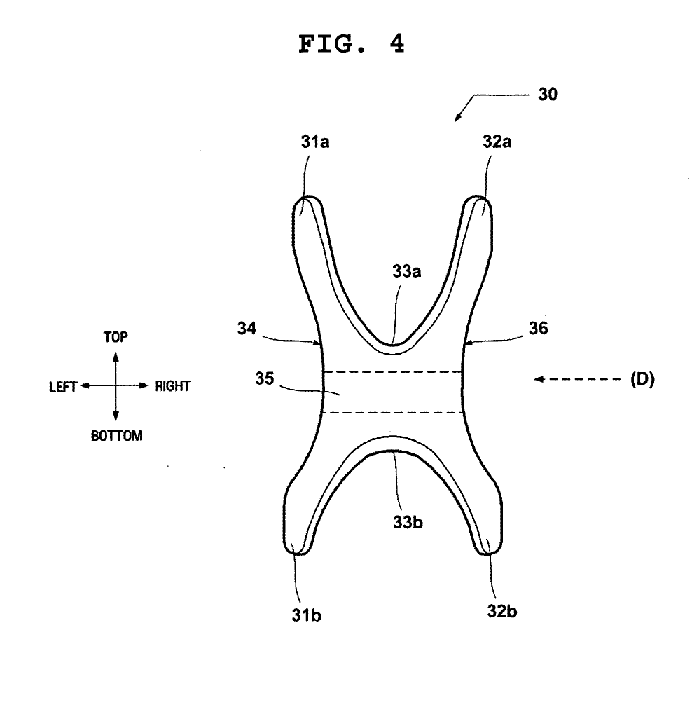

The through-hole 35 is a hole bored through left and right portions of the spacer 30 and has an elongated slot shape which is wide enough to receive the width of a strap. A detailed description of the structure and shape of the through-hole 35 will be provided with reference to FIG. 5.

FIG. 5 shows the spacer 30 of FIG. 4 as viewed from the right side (D) of FIG. 4. As shown in FIG. 5, the right portion of the spacer 30 is not symmetric about the top-bottom axis. As such, when the spacer 30 is inserted between two adjacent spinous processes in the (E) direction, the first and second notches 33a and 33b can be fitted with the two spinous processes. Considering the general shape of spinous processes, the first and second notches 33a and 33b may have steeper slopes from an insertion front part to an insertion rear part.

The through-hole 35 is bored through left and right portions of the spacer 30, and has a width b which is wide enough to receive the width of a strap 40. The through-hole 35 has a height h that allows the injection of three strands of the strap, i.e., that barely allows passage of the three strands of the strap. That is, since a predetermined frictional force must be generated among the strands of the strap passed through the through-hole 35, compression of the strap is required. Thus, it is preferable that the through-hole 35 is formed to a height equal to three times of the thickness of the strap minus a predetermined value (α). The larger the a value, the smaller the frictional force among the strands of the strap, whereas the smaller the a value, the smaller the frictional force among the strands of the strap. In this regard, the a value is determined according to a desired frictional force among the strands of the strap. The a value can be determined empirically, but it may be substantially similar to the thickness of the strap. Preferably, the spacer 30 is made of an inert and solid metal such as titanium.

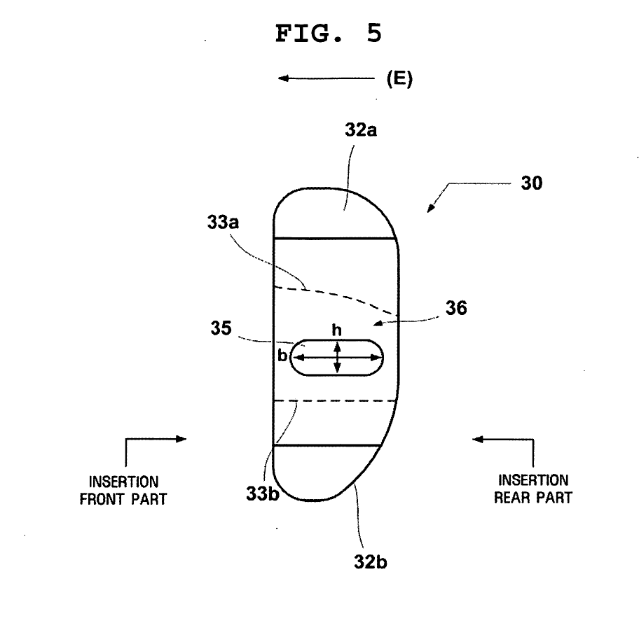

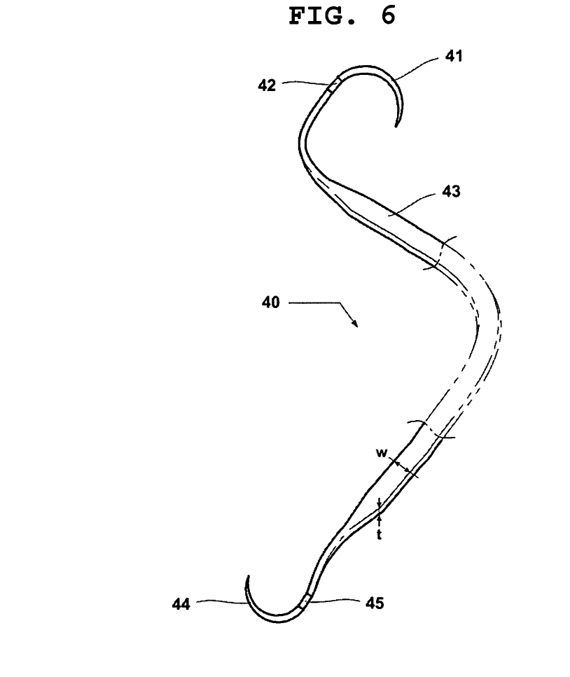

FIG. 6 is a perspective view illustrating a strap 40 constituting an intervertebral implant according to the present invention. The strap 40 binds two adjacent spinous processes and a spacer in a FIG. 8 form. The strap 40 includes a band 43 for fastening the spacer, a hooks formed at both ends of the band 43, and connecting portions 42 and 45 connecting the hook to the band 43.

There are two hooks: a first hook 41 and a second hook 44. The first hook 41 passes through an interspinous ligament above an upper spinous process and the shape of the first hook 41 is determined by the upper shape of the upper spinous process. The second hook 44 passes through an interspinous ligament below a lower spinous process, and the shape of the second hook 44 is determined by the lower shape of the lower spinous process. Thus, the dimensions and radius of curvature of the first hook 41 are generally larger than those of the second hook 44.

The band 43 has a relatively large width w and a relatively small thickness t. Both ends of the band 43 have a relatively narrow width to connect the band 43 to the first and second hooks 41 and 44. The band 43 may be made of a material with a certain coefficient of friction, and which is harmless to the human body, for example, a synthetic fiber made of polyester, natural leather, artificial leather, or the like.

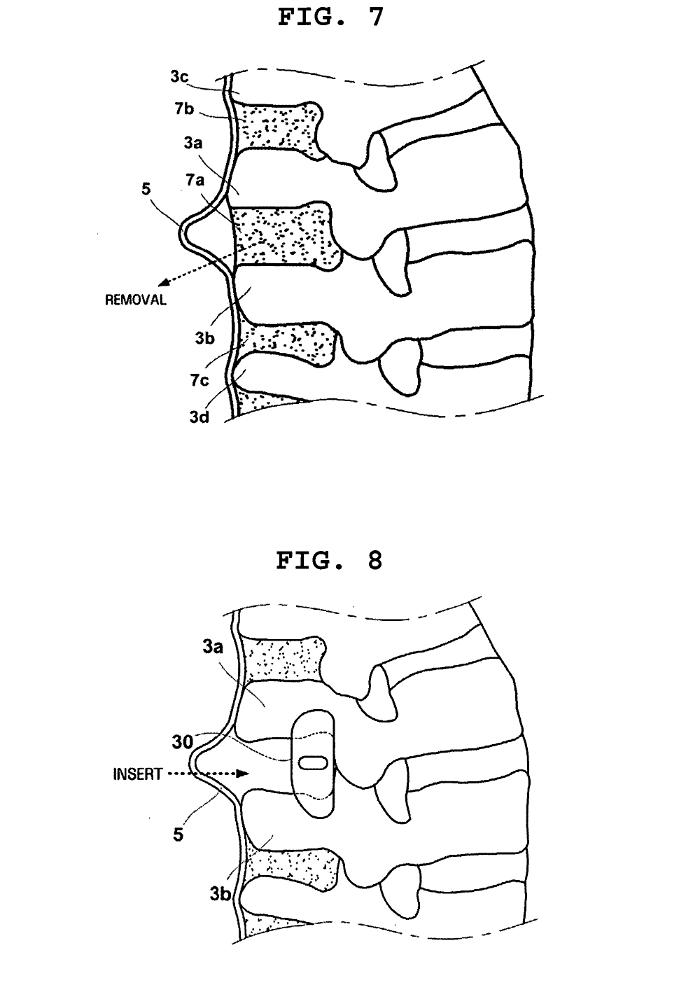

FIGS. 7 and 8 are views illustrating the insertion of a spacer 30 into the body of a patient. First, as shown in FIG. 7, a supraspinous ligament 5 is lifted and its corresponding interspinous ligament 7a is removed. Then, as shown in FIG. 8, the spacer 30 is inserted in a space between two adjacent spinous processes 3a and 3b where the interspinous ligament 7a has been removed.

FIG. 9 shows the spacer 30 inserted between the spinous processes 3a and 3b through the procedure shown in FIGS. 7 and 8 as viewed from the back of the body. As shown in FIG. 9, the spacer 30 is inserted between the two spinous processes 3a and 3b, and the two spinous processes 3a and 3b are held by opposing notches 33a and 33b of the spacer 30. However, each spinous process may be slightly moved left or right by flexion of the spinal column. As such, the spacer 30 must be installed with a strap. For convenience of illustration, the securing of a spacer with a strap will be described with reference to (F) of FIG. 9.

Two embodiments about the securing of a spacer with a strap according to the present invention will be provided hereinafter.

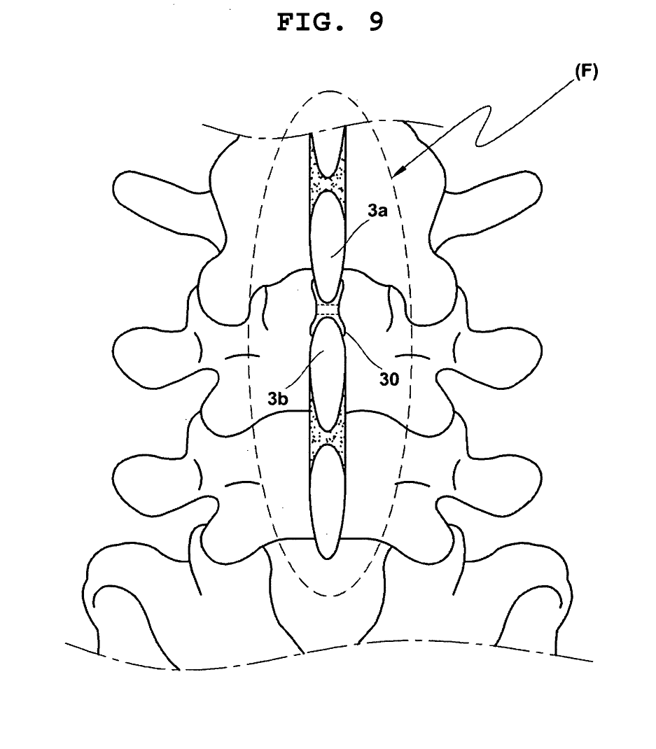

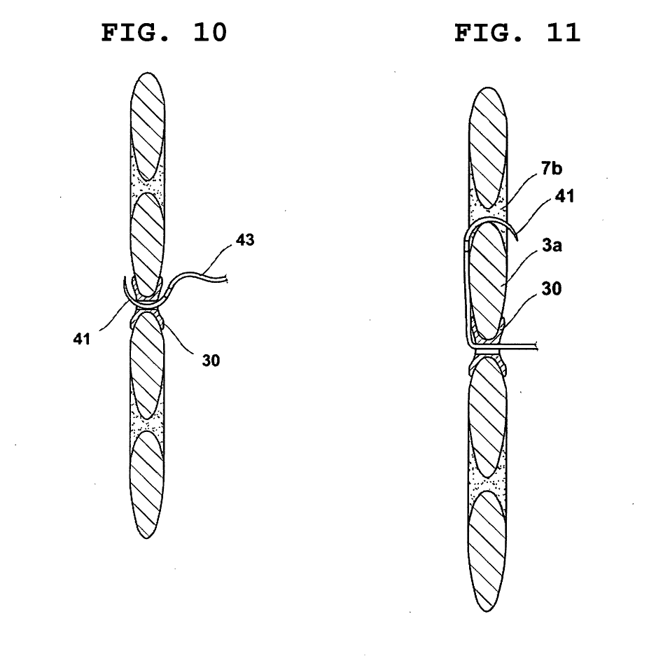

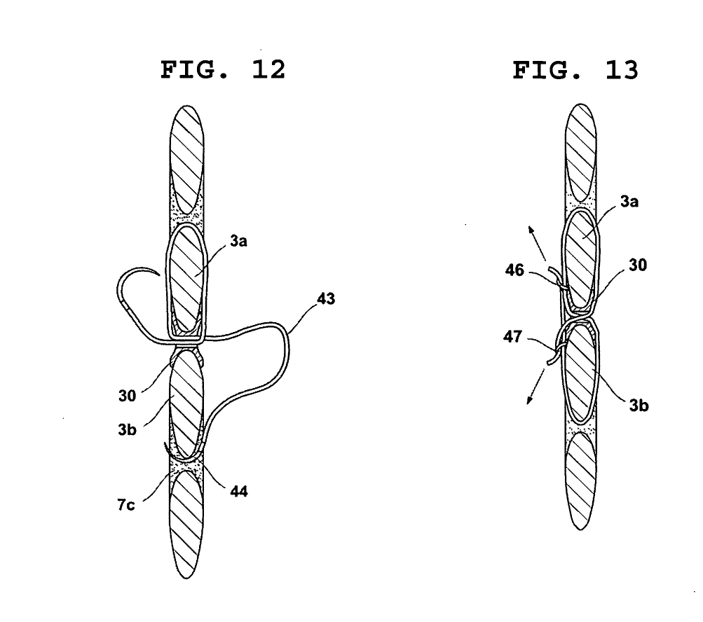

FIGS. 10 through 13 illustrate the securing of a spacer with a strap according to a first embodiment of the present invention. First, referring to FIG. 10, a first hook 41 is allowed to pass through a through-hole 35 (not shown) of a spacer 30. Then, referring to FIG. 11, the first hook 41 is allowed to pass through an area of an interspinous ligament 7b near an upper portion of an upper spinous process 3a. Referring to FIG. 12, the first hook 41 is again allowed to pass through the through-hole 35 of the spacer 30, and a second hook 44 is allowed to pass through an area of an interspinous ligament 7c near a lower portion of a lower spinous process 3b.

Finally, the first hook 41 and the second hook 44 are removed, and both ends of a band 43 are tightly pulled in the direction of the arrow shown in FIG. 13. When both ends of the band 43 are pulled, the band 43 is closely contacted to depressions 34 and 36 (not shown) of the spacer 30, which ensures a more secure fastening of the spacer 30 to the upper and lower spinous processes 3a and 3b. A knot 46 is tied with the first hook-free end of the band 43 and a strand of the band 43 surrounding the lower spinous process 3b, and a knot 47 is tied with the second hook-free end of the band 43 and a strand of the band 43 surrounding the upper spinous process 3a.

According to the above-described embodiment, the spacer 30 is secured to the upper and lower spinous processes 3a and 3b by the knots 46 and 47 tied using both ends of the band 43. However, securing of a spacer with a band according to another embodiment (as will be described later) is accomplished using band knots and friction between the strands of the band.

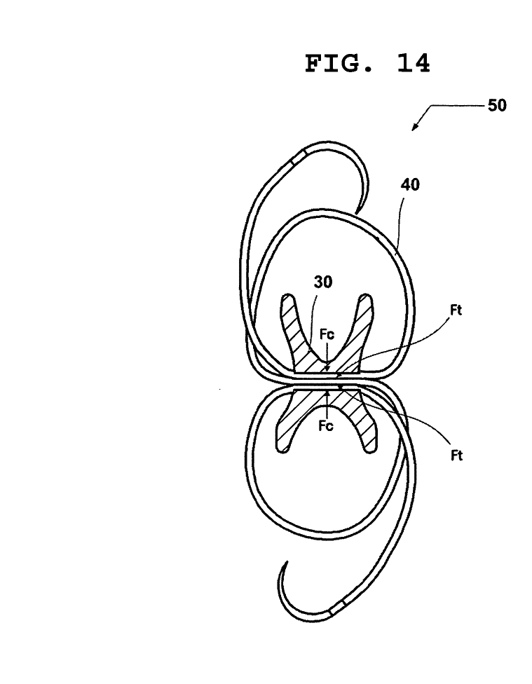

FIG. 14 illustrates a spacer 30 installed with a strap 40 according to a second embodiment of the present invention. Referring to FIG. 14, when a band 43 of the strap 40 passes through a through-hole 35 three times in a FIG. 8, a compression force Fc is produced in the direction depicted by the arrows of FIG. 14. The compression force Fc also acts between adjacent strands of the band 43. A frictional force Ft is generated between the strands of the band 43 and is proportional to the compression force Fc and the coefficient of friction of the strands of the band 43. Of course, a frictional force is also generated between an inner surface of the through-hole 35 and the band 43. However, since the frictional force generated between the inner surface of the through-hole 35 and the band 43 is much smaller than the frictional force Ft generated between the strands of the band 43, only the frictional force Ft generated between the strands of the band 43 will be considered. According to another embodiment, the frictional force between the inner surface of the through-hole 35 and the band 43 can be adjusted to be higher than the frictional force Ft between the strands of the band 43. For this, the band 43 and the through-hole 35 can be structurally modified in an appropriate manner.

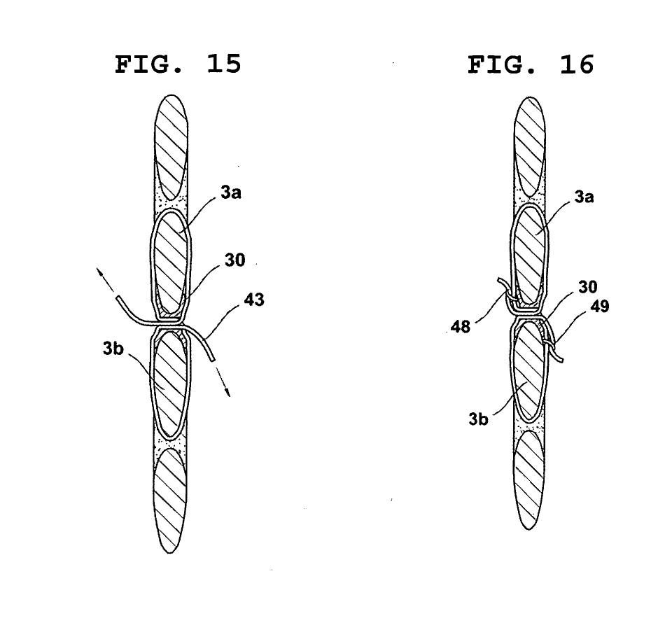

FIGS. 15 and 16 illustrate the installing of the spacer 30 with the strap 40 according to the second embodiment of the present invention. Subsequent processes to those shown in FIGS. 10 through 12 are illustrated in FIGS. 15 and 16. Thus, as in the first embodiment, the processes shown in FIGS. 10 through 12 are first performed in the second embodiment.

Referring to FIGS. 10 through 12 and 14 through 16, first, the first hook 41 is inserted to pass through the through-hole 35 of the spacer 30. Then, the first hook 41 is passed through an area of the upper interspinous ligament 7b near the upper portion of the upper spinous process 3a. The first hook 41 is again passed through the through-hole 35 of the spacer 30, and the second hook 44 is passed through an area of the lower interspinous ligament 7c near the lower portion of the lower spinous process 3b.

Then, like the first hook 41, the second hook 44 is again passed through the through-hole 35. Thereafter, the first and second hooks 41 and 44 connected to both ends of the band 43 are removed and both the ends of the band 43 are tightly pulled in the directions shown by the arrows of FIG. 15. By doing so, the motion of the band 43 is retarded by the frictional force between the strands of the band 43, and the upper and lower spinous processes 3a and 3b are stably held in notches of the spacer 30. In this way, when the motion of the band 43 is retarded by the frictional force between the strands of the band 43, the band 43 is closely contacted to depressions 34 and 36 of the spacer 30, which ensures a more secure fastening of the spacer 30 to the upper and lower spinous processes 3a and 3b.

Finally, knots 48 and 49 are tied using both ends of the band 43 and the strands of the band 43 surrounding the upper and lower spinous processes 3a and 3b. The securing of the spacer 30 to the upper and lower spinous processes 3a and 3b using the band 43 is accomplished mainly by the frictional force between the strands of the band 43 and it is assisted by the knots 48 and 49.

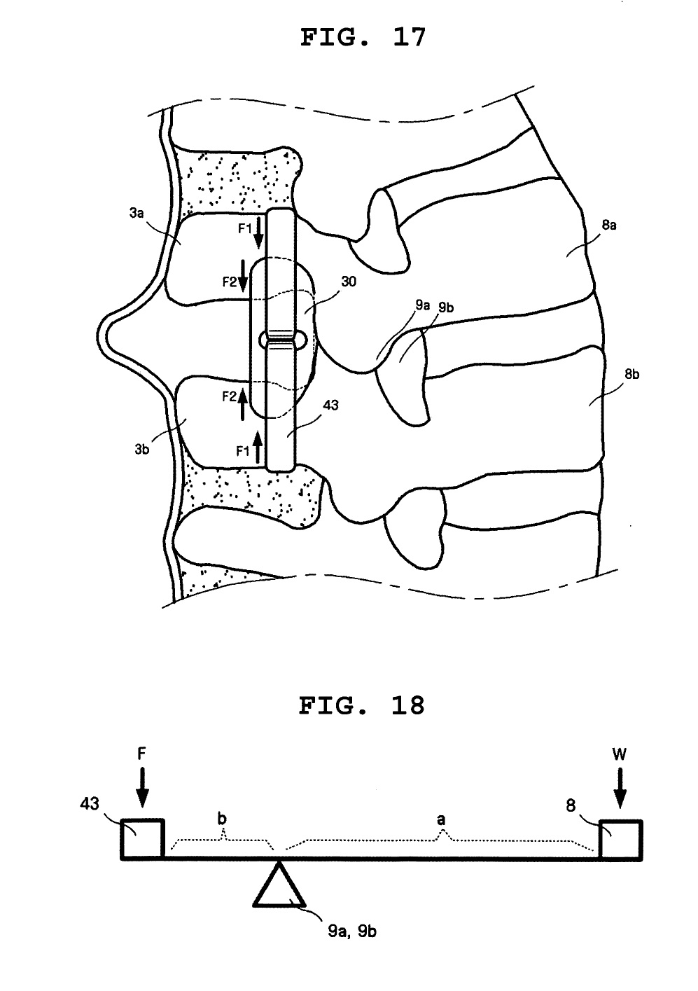

FIG. 17 is a lateral view of a spinal column showing an intervertebral implant of a spacer-band complex inserted between two adjacent spinous processes 3a and 3b according to an embodiment of the present invention. Referring to FIG. 17, a spacer 30 maintains a predetermined space between the two spinous processes 3a and 3b (predetermined distance between notches), and supports a compression force F2 acting between the two spinous processes 3a and 3b. A band 43 integrally binds the spacer 30 and the two spinous processes 3a and 3b to produce a predetermined bearing force F1 on the two spinous processes 3a and 3b so that a widening of an interspinous space is prevented. In this way, when the widening of the interspinous space is prevented by the band 43, several problems such as spinal stenosis, which is due to narrowing of the space between corresponding vertebral bodies 8a and 8b, can be corrected.

That is, an external load is dispersed to vertebral bodies and facet joints in an appropriate ratio, thereby reducing the pressure applied to the facet joints and the pressure applied to intervertebral discs. Therefore, the intervertebral discs can be perfectly preserved and an intervertebral space can be appropriately maintained. Thus, the intervertebral implant according to the present invention can prevent various diseases caused due to narrowing of the intervertebral space as well as various diseases caused due to narrowing of the interspinous space.

Physical Support

In the intervertebral implant according to the present invention, the band 43 also serves to prevent a relative displacement between a superior facet 9b and an inferior facet 9a. That is, the band 43 is also responsible for facet joint fastening. Likening the human spinal column to a lever system, the inferior and superior facets 9a and 9b serve as the fulcrum, the vertebral bodies 8a and 8b serve as the load to be moved, and the muscles do the work. In particular, fastening the inferior and superior facets 9a and 9b (acting as the fulcrum) in order to prevent damage, is an important factor in the proper operation of the spinal column. The fastening of the facet joints can eliminate the pain caused by the movement of the facet joints, reduce surgical aftereffects, and prevent spinal stenosis (by limiting the motion in the facet joints).

To model the spinal column in terms of a lever system, a first class lever system is illustrated in FIG. 18. The band 43 is positioned in the place where the force needs to be applied and it produces the force F1 in order to compress the two spinous processes 3a and 3b. By means of the force F1, lordosis occurs in the sagittal section of the spinal column. At this time, extensor muscles are pulled and thus the lever arm length is increased. As a result, the force F1 balances the load W applied to the vertebral bodies 8a and 8b by means of the inferior and superior facets 9a and 9b acting as the fulcrum. Here, the extensor muscles offset the external moment of the torso, and generate a posterior shear force offsetting an anterior shear force generated by an upper vertebral body or an external load.

The load W applied to the vertebral bodies 8a and 8b is mainly determined by the muscular force of the extensor muscle (erector spinae muscle) of the lower back. As such, this system can be explained by describing the action of the muscular force on the vertebral bodies. According to the formula: F×a=W×b, as the length of the extensor muscle corresponding to the lever arm increases, the action range of the extensor muscle on the vertebral bodies increases.

When two spinous processes are compressed by ligamentoplasty according to the present invention, the normal lumbar lordosis is restored and thus the length of the extensor muscle, i.e., the lever arm increases. If the length of the lever arm increases, the action range of the muscular force increases, and the load acting on the vertebral bodies can be offset accordingly.

FIGS. 19 through 21 are diagrammatic views illustrating the actions of intervertebral implants according to a conventional technique and the present invention. FIG. 19 is a diagrammatic view illustrating a normal state of vertebrae and facet joints. Referring to FIG. 19, facet joints 9a and 9b act as the fulcrum of a spinal lever. Upper and lower spinous processes 3a and 3b and upper and lower vertebral bodies 8a and 8b are respectively positioned at both ends of the facet joints 9a and 9b. In FIG. 19, the interspinous space and the intervertebral space are normally maintained.

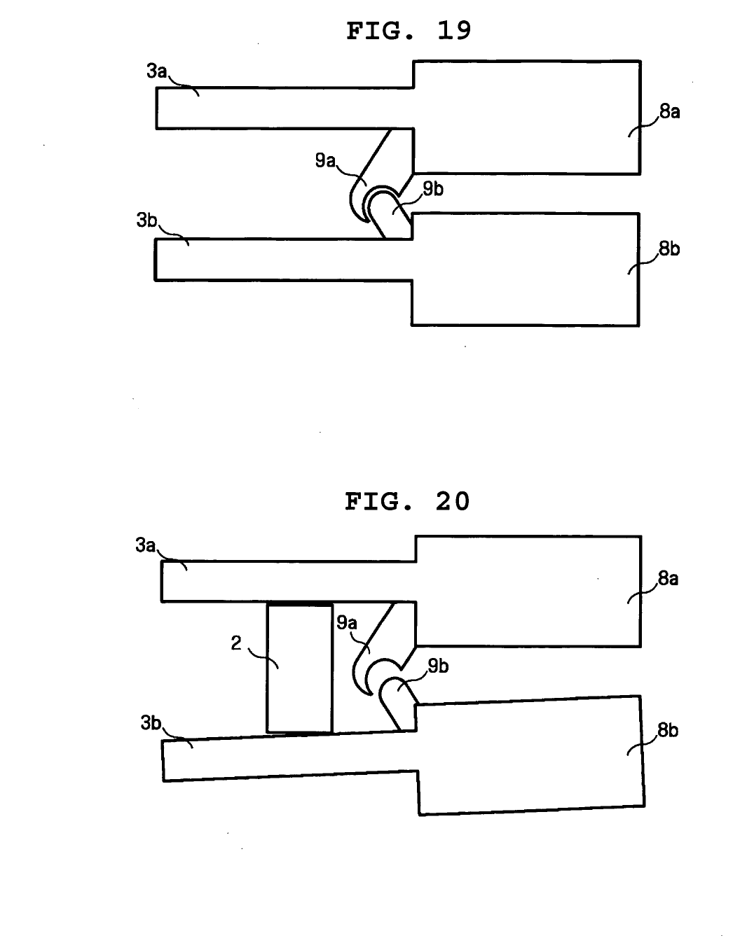

FIG. 20 is a diagrammatic view showing the state of a spinal column after a spacer is inserted therein according to a conventional technique. In this case and in the conventional technique shown in FIG. 2 in which the upper and lower spinous processes 3 and 3b and the spacer 2 are bound with the ties 13a and 13b, since the upper and lower spinous processes 3a and 3b and the spacer 2 are not integrally fastened, a relative displacement between the upper spinous process 3a and the lower spinous process 3b may occur. Furthermore, when a predetermined force acts on facet joints 9a and 9b serving as the fulcrum of a spinal lever, a relative displacement between the facet joints 9a and 9b may occur, thereby causing various diseases associated with degeneration of the fulcrum. As a result, the spinal lever may not work properly. In addition, a slight widening of the interspinous space may occur. In this case, since the intervertebral space is decreased, a disease such as spinal stenosis may become worse after surgery.

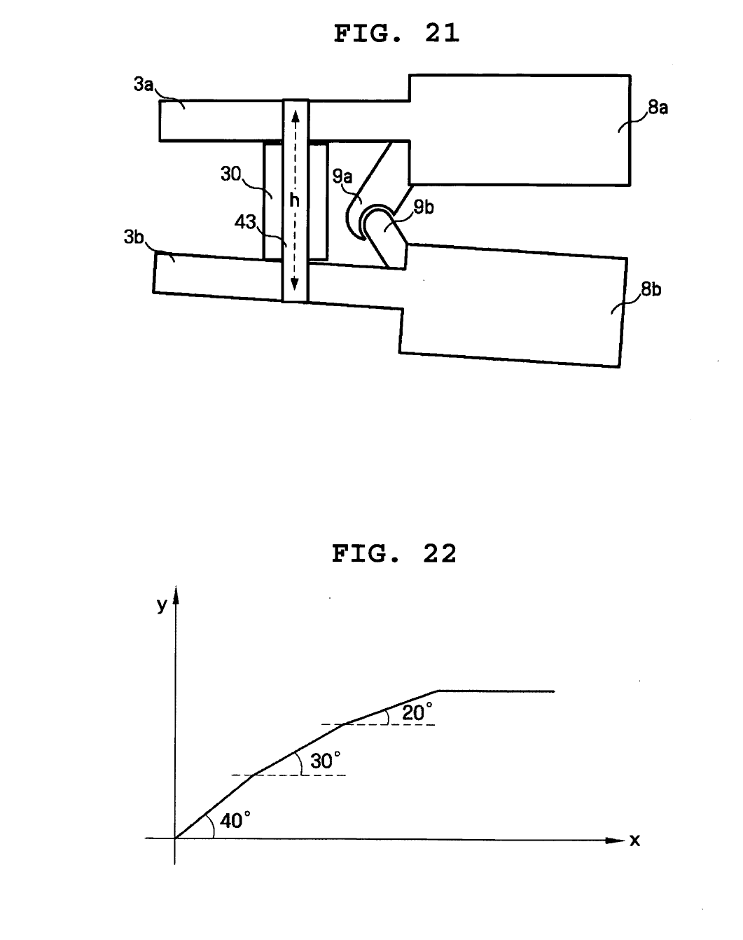

FIG. 21 is a diagrammatic view showing the state of a spinal column after a spacer is inserted therein according to the present invention. Referring to FIG. 21, a spacer 30 is securely fastened to upper and lower spinous processes 3a and 3b with a band 43. Therefore, a relative displacement between facet joints 9a and 9b does not occur, and thus the spinal lever works properly even after surgery. Furthermore, various diseases arising in the facet joints 9a and 9b when the facet joints 9a and 9b are moved or impacted can be prevented.

In addition, even when the intervertebral space narrows, i.e., spinal stenosis occurs, the vertebral bodies 8a and 8b can be separated from each other by more than the predetermined space by strengthened muscular bearing power. Further, the normal vertebral lordosis can be restored by fastening with the band 43. However, considering individual differences, it is preferable to adjust the height h shown FIG. 21 so as not to cause local kyphosis.

Clustering for Analysis

Hereinafter, clustering for fitting a first notch 33a to a lower surface of an upper spinous process 3a will be described in detail. It is well known that individuals have different body structures, and the shapes of spinous processes may also be diversely distributed. Thus, custom manufacture of an intervertebral implant and corresponding surgical treatment according to the shapes of the spinous processes of each individual are almost impossible. In this regard, there is need to perform clustering for the shape of the first notch 33a using statistical data to obtain a predetermined number of first notch shapes.

Of course, clustering for the shape of a second notch 33b can also be performed in the same way. Generally, however, the shape of the second notch 33b is not diverse and the profile of the second notch 33b can be considered a horizontal straight line. Thus, clustering for the second notch 33b is not profitable. In this regard, the present invention will be described in terms of clustering for the first notch 33a.

Referring to FIG. 5, the profile of the first notch 33a has a shape in which a plurality of points are connected by line segments of different functions. For example, the profile of the first notch 33a may not be composed of a single straight line with a slope of 20° but may be composed of three straight line segments with different slopes of 40°, 30°, and 20° joined at three inflection points. Of course, the profile of the first notch 33a may also have a shape in which a plurality of points are connected by curved lines with different predetermined curvatures.

In particular, considering the lower surface shape of an upper spinous process 3a to be supported by the first notch 33a, the profile of the first notch 33a has a more gently sloped shape from the insertion rear part to the insertion front part.

FIG. 22 is a graph illustrating the profile slope of a first notch of an intervertebral implant according to an embodiment of the present invention plotted in the x-y plane. Referring to FIG. 22, assuming that the end of the first notch 33a near the insertion rear part is the origin in the x-y plane, the profile of the first notch 33a is initially sloped at an angle of 40°. However, at predetermined points the slope changes to angles of 30°, 20°, and 0°.

An available profile slope range for the first notch 33a is provided by measuring lower surface slopes of spinous processes intended for spacer insertion in a large number of patients and then statistically analyzing the data. In an embodiment of the present invention, angles between the third and fourth spinous processes (referred to as “L34,” hereinafter) and between the fourth and fifth spinous processes (referred to as “L45,” hereinafter) where the intervertebral implant is mainly inserted are measured. Precisely speaking, the lower surface slope of the third spinous process of L34 is measured and the lower surface slope of the fourth spinous process of L45 is measured.

Lower surface slope measurements for the third spinous process are presented in Table 1 below. For this, 10 computed tomography (CT) images were obtained from 103 spinal stenosis patients by axial CT scan. The lower surface slope measurements listed in Table 1 were obtained using CT images of the third spinous process.

TABLE 1

| Section | 1 | 2 | 3 | 4 | 5 | 6 | 7 | 8 | 9 | 10 | 11 |
|---|---|---|---|---|---|---|---|---|---|---|---|
| 1 | 17 | 27.2 | 10.2 | 20.4 | 23.8 | 23.8 | 23.8 | 27.2 | 27.2 | 20.4 | 10.2 |
| 2 | 20.4 | 20.4 | 20.4 | 17 | 13.6 | 30.6 | 37.4 | 23.8 | 27.2 | 20.4 | 27.2 |
| 3 | 17 | 23.8 | 17 | 20.4 | 13.6 | 27.2 | 30.6 | 20.4 | 27.2 | 34 | 20.4 |
| 4 | 17 | 17 | 20.4 | 20.4 | 17 | 27.2 | 13.6 | 27.2 | 27.2 | 30.6 | |
| 5 | 23.8 | 23.8 | 17 | 27.2 | 23.8 | 23.8 | 23.8 | 27.2 | 27.2 | 13.6 | |
| 6 | 27.2 | 23.8 | 17 | 17 | 27.2 | 10.2 | 27.2 | 27.2 | 23.8 | 20.4 | |
| 7 | 10.2 | 20.4 | 20.4 | 13.6 | 40.8 | 20.4 | 13.6 | 17 | 13.6 | 20.4 | |
| 8 | 13.6 | 27.2 | 23.8 | 17 | 13.6 | 23.8 | 27.2 | 27.2 | 23.8 | 6.8 | |
| 9 | 20.4 | 23.8 | 19.7 | 23.8 | 27.2 | 23.8 | 23.8 | 17 | 27.2 | 27.2 | |
| 10 | 13.6 | 23.8 | 13.6 | 0 | 20.4 | 6.8 | 23.8 | 37.4 | 27.2 | 30.6 | |

Lower surface slope measurements for the fourth spinous process are presented in Table 2 below. For this, 10 CT images were obtained from 103 spinal stenosis patients by axial CT scan. The lower surface slope measurements listed in Table 2 were obtained using CT images containing the fourth spinous process.

TABLE 2

| Section | 1 | 2 | 3 | 4 | 5 | 6 | 7 | 8 | 9 | 10 | 11 |
|---|---|---|---|---|---|---|---|---|---|---|---|
| 1 | 0 | 20.4 | 3.4 | 17 | 0 | 20.4 | 34 | 23.8 | 17 | 10.2 | 10.2 |
| 2 | 17 | 0 | 17 | 13.6 | 6.8 | 30.6 | 27.2 | 13.6 | 20.4 | 13.6 | 23.8 |
| 3 | 3.4 | 17 | 10.2 | 17 | 20.4 | 30.6 | 27.2 | 17 | 17 | 13.6 | 13.6 |
| 4 | 3.4 | 13.6 | 20.4 | 13.6 | 20.4 | 13.6 | 10.2 | 17 | 13.6 | 13.6 | |
| 5 | 17 | 20.4 | 27.2 | 23.8 | 17 | 17 | 17 | 23.8 | 23.8 | 10.2 | |
| 6 | 20.4 | 23.8 | 6.8 | 13.6 | 20.4 | 3.4 | 3.4 | 20.4 | 10.2 | 20.4 | |
| 7 | 13.6 | 13.6 | 13.6 | 10.2 | 27.2 | 23.8 | 10.2 | 30.6 | 13.6 | 13.6 | |
| 8 | 17 | 17 | 17 | 20.4 | 10.2 | 30.6 | 34 | 13.6 | 6.8 | 10.2 | |
| 9 | 17 | 10.2 | 13.6 | 17 | 23.8 | 17 | 17 | 34 | 30.6 | 17 | |
| 10 | 6.8 | 10.2 | 13.6 | 0 | 13.6 | 6.8 | 13.6 | 0 | 17 | 23.8 | |

As shown in Tables 1 and 2, L34 and L45 have a different lower surface slope distributions. That is, with respect to L45, a lower surface slope distribution from 0° to 15° is relatively high.

Table 3 presents lower surface slope measurements of the third and fourth spinous processes. The lower surface slopes of the third and fourth spinous processes of 50 spinal stenosis patients were measured by X-ray. In lower surface slope CT measurements scan as shown in Tables 1 and 2, a blurring effect may appear in CT images. Also, converted slopes based on sliced images may be slightly inaccurate. X-ray imaging can exclude these possibilities.

TABLE 3

| | Lower surface slope of the | | | | | Lower surface slope of the | | | | |
|---|---|---|---|---|---|---|---|---|---|---|
| Section | third spinous process | | | | | fourth spinous process | | | | |
| 1 | 12 | 29.3 | 9.7 | 25.3 | 20.4 | 0 | 36 | 6.3 | 17.3 | −5 |
| 2 | 34 | 26.6 | 37 | 0 | 12.5 | 24 | 3.3 | 16 | 0 | 11 |
| 3 | 0 | 6 | 22.3 | 18 | 0 | 3 | 4.1 | 22.1 | 29.5 | 10.7 |
| 4 | 5.5 | 20 | 11.1 | 19.3 | 26 | 12.5 | 12 | 8.3 | 3.7 | 5.6 |
| 5 | 35 | 0 | 11.4 | 6.1 | 23.3 | 8 | 0 | 0 | 2.1 | 18 |
| 6 | 25.3 | 24 | 17.3 | 0 | 16.2 | 0 | 11 | 4.3 | 14 | 2 |
| 7 | 15 | 25 | 0 | 0 | 24 | 12 | 0 | 2.3 | 0 | 0 |
| 8 | 11.4 | 19 | 29.3 | 18.6 | 1 | 4 | 22.6 | 21 | 11.4 | 0 |
| 9 | 13.4 | 34 | 35.9 | 14 | 21 | 4.5 | 6 | 8 | 6.5 | 0 |
| 10 | 24.6 | 14 | 7 | 13.6 | 8.2 | 7 | 20 | 12.3 | 9.3 | 3 |

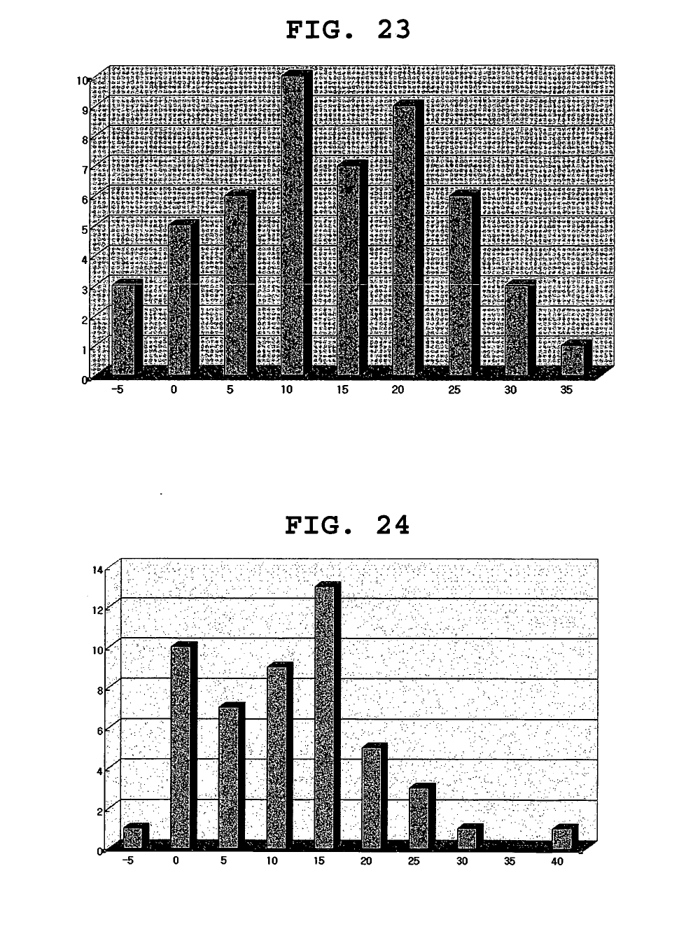

FIG. 23 is a histogram illustrating the distribution of the lower surface slope measurements of the third spinous process. The distribution of the lower surface slope measurements of the third spinous process is represented by dividing the lower surface slope measurements of the third spinous process presented in Table 3 into groups separated by 5°.

Referring to FIG. 23, the distribution of the lower surface slope measurements of the third spinous process approximates a normal curve. In particular, 26 patients are distributed in a slope range of 15° to 25°.

FIG. 24 is a histogram illustrating the distribution of the lower surface slope measurements of the fourth spinous process. The distribution of the lower surface slope measurements of the fourth spinous process is represented by dividing measurements presented in Table 3 into groups separated by 5°.

Referring to FIG. 24, the lower surface slope measurements of the fourth spinous process exhibit a left-skewed distribution. In particular, 39 patients are distributed in a slope range of 0° to 15°.

As shown in FIGS. 23 and 24, the lower surface slope characteristics of the third spinous process are different from those of the fourth spinous process. A currently available spacer is mainly inserted into L34 or L45 and is used regardless of where it is inserted.

In this respect, a first notch with a profile slope of 20° can be generally used for L34 but it may need to be greatly modified for L45. Furthermore, when a spacer having a first notch with a profile slope of 20° is inserted between L34 of a patient having a L3 lower surface slope greatly deviated from 20°, the vertical bearing power of the spacer for L34 may be lowered. The spacer 30 according to the above-described embodiment of the present invention is more adaptable when numerical analysis is used to connect a plurality of statistically obtained points of the profile by line segments of different functions.

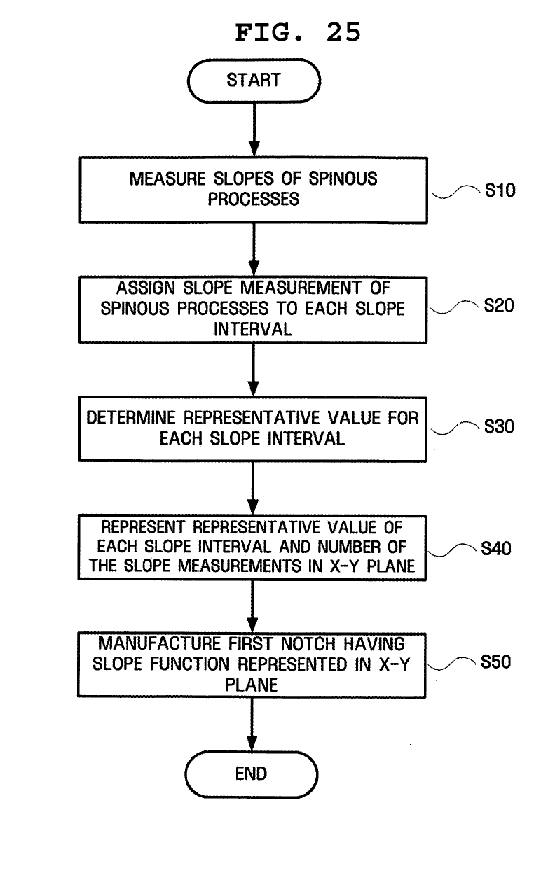

FIG. 25 is a flow diagram illustrating a method of manufacturing a first notch of an intervertebral implant according to an embodiment of the present invention.

Referring to FIG. 25, first, a predetermined sample group is drawn from a population containing a plurality of patients, and lower surface slopes of spinous processes of the members of the sample group are measured (operation S10). This embodiment will be described in terms of the lower surface slopes of the third and fourth spinous processes presented in Table 3, which contains data of a sample group containing 50 members.

The lower surface slope measurements of the spinous processes are assigned to predetermined slope intervals (operation S20). In this embodiment, the slope interval is 10°. However, the slope interval can be optionally determined by one of ordinary skill in the art. The slope interval of 10° in this embodiment is provided for convenience of illustration. However, it is preferable to gradually shorten the slope intervals to obtain a substantial curve. That is, a plurality of statistically obtained points may be connected to form a curve by polynomial interpolation, spline interpolation, or alternatively they may be connected as a substantial curve by straight line segments with shortened slope intervals.

Table 4 shows a 10 degree slope interval for the lower surface slope measurements of the third and fourth spinous processes of Table 3 (50 member sample group). In particular, L345 represents a mean value of the lower surface slope measurements of the third and fourth spinous processes. That is, L345 is a mean lower surface slope value of the third and fourth spinous processes, which is calculated in order to produce a spacer that can be inserted into both L34 and L45.

TABLE 4

| Section | L34 | L45 | L345 (=(L34 + L45)/2) |
|---|---|---|---|
| 40°˜50° | 1 | 1 | 1 |
| 30°˜40° | 9 | 1 | 1 |
| 20°˜30° | 16 | 8 | 8 |
| 10°˜20° | 16 | 22 | 22 |
| 0°˜10° | 8 | 17 | 17 |
| −10°˜0° | 0 | 1 | 1 |

After the slope measurements of the spinous processes are assigned to each slope interval, a representative value for each slope interval is determined (operation S30). The representative value for each slope interval may be the lower limit value, the upper limit value, or a mean value of each slope interval, or it can be optionally selected by one of ordinary skill in the art. For example, in this embodiment, the lower limit value of each slope interval is used as the representative value. Thus, the representative values for the slope intervals are 40°, 30°, 20°, 10°, 0°, and −10°. The operation of determining the representative value S30 may also be followed by the operation of assigning the slope measurements to each slope interval S20.

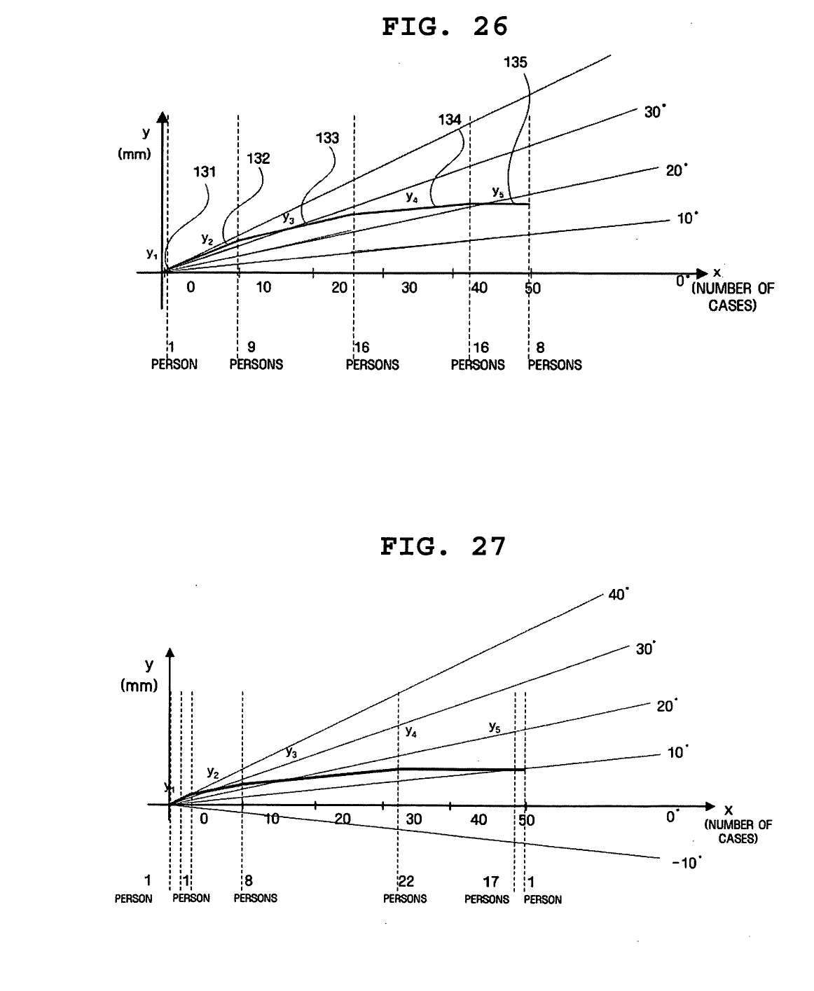

The representative value of each slope interval and the number of the slope measurements assigned to each slope interval are represented in the x-y plane (operation S40). The x-axis is the cumulative number of the assigned slope measurements and the y-axis is the height of the first notch. FIG. 26 illustrates the lower surface slope of the third spinous process plotted in the x-y plane.

Referring to FIG. 26, the cumulative number of the assigned slope measurements is represented by the x-axis. The number 131 represents a line segment with a slope of 400 between the origin and the point where the cumulative number of the assigned slope measurements in a 40-50 degree interval is 1, 132 represents a line segment with a slope of 30° between the end point of the line segment 131 and the point where the cumulative number of the assigned slope measurements in a 30-40 degree interval is 10 (1+9), and 133 represents a line segment with a slope of 20° between the end point of the line segment 132 and the point where the cumulative number of the assigned slope measurements in a 20-30 degree interval is 26 (10+16). Numbers 134 and 135 are line segments with a slope of 10° and 0°, respectively, plotted in the same manner as the above line segments.

The above line segments can be represented by Equation 1.

$$y = {\sum }_{m = 1}^{n} \{ tan {θ}_{m} ( x - {N}_{m - 1} ) + tan {θ}_{m - 1} {N}_{m - 1} \} * u ( {N}_{m - 1} , {N}_{m} ) \\ {N}_{0} = 0 , {θ}_{0} = 0 \qquad ( 1 )$$

where y is a function composed of straight-line segments with different slopes obtained by connecting a plurality of statistically obtained points, x is the number of assigned slope measurements, n is a n-th interval, Nₙ is the cumulative number of assigned slope measurements in an n-th interval, θn is a representative value of an n-th interval, u(Nₙ₋₁, Nₙ) is a uniform function having a value of 1 in the interval (Nₙ₋₁, Nₙ), and N₀ and θ₀ are each zero.

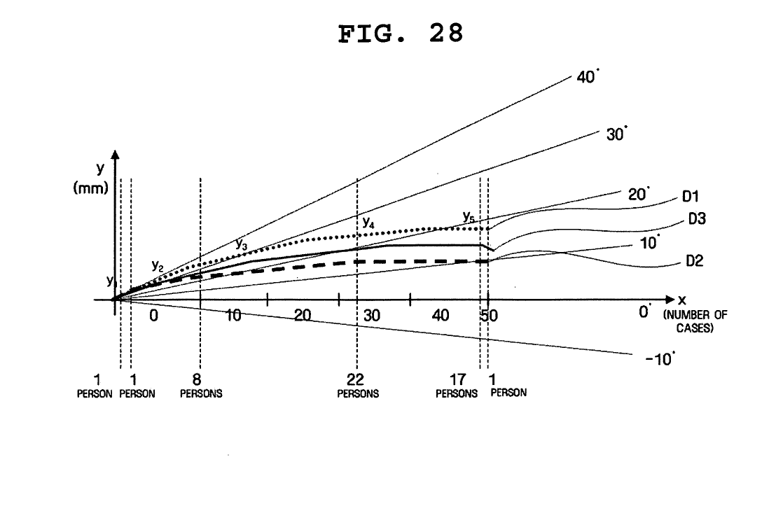

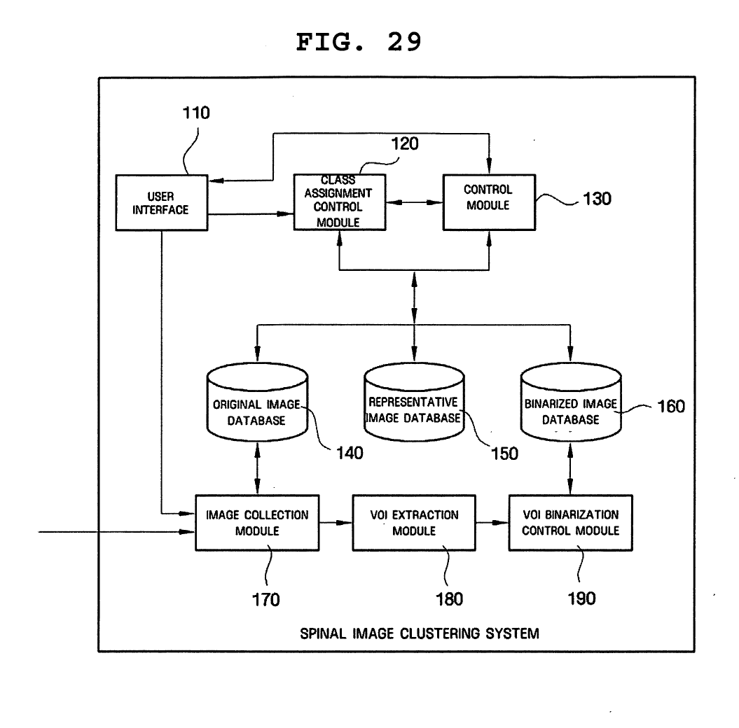

FIG. 27 is a graph illustrating the lower surface slope of the fourth spinous process plotted in the x-y plane. FIG. 28 is a graph illustrating a mean lower surface slope of the third and fourth spinous processes plotted in the x-y plane. The mean lower surface slope of the third and fourth spinous processes can also be plotted in the x-y plane in the same manner.

In FIG. 28, D1 is a graph of a function for L34 of FIG. 26, D2 is a graph of a function for L45 of FIG. 27, and D3 is a graph of a function for L345. Referring to FIG. 28, it can be seen that D3 is plotted between D1 and D2. Advantages of the function for L345 will be described later.

Finally, first notches 33a having the same profile slopes as the slopes of the functions obtained in FIGS. 26 through 28 are manufactured (operation S50). According to the above-described method, there can be manufactured an intervertebral implant having a spacer with a first notch having a profile slope obtained by connecting a plurality of points by line segments of different functions.

As described above, a first notch 33a supports a lower portion of an upper spinous process 3a. Thus, when the profile slope of the first notch 33a is the same as the lower surface slope of the upper spinous process 3a, the bearing power for a vertical-directed compression force increases. As in the above-described embodiment of the present invention, when a first notch 33a is composed of a plurality of planes having different slopes, an intervertebral implant including a spacer with the first notch 33a can be generally applied to upper spinous processes having different lower surface slopes.

Furthermore, since a first notch of the present invention is manufactured based on the lower surface slope measurements of spinous processes of a plurality of spinal stenosis patients, a slope interval to which a larger number of patients are statistically assigned is in a higher ratio, a bearing power for a vertical-directed compression force can be enhanced, as compared to a first notch having a uniform distribution of different profile slopes. Also, an intervertebral implant including a spacer with such a first notch 33a can be applied to more patients.

In addition, a spacer for L34 and a spacer for L45 can be separately manufactured. Alternatively, a spacer that can be commonly inserted into L34 and L45 can also be manufactured. Therefore, trimming of a first notch 33a during surgery or partial removal of a lower portion of an upper spinous process 3a may not be required.

In the above-described embodiment, a plurality of points are connected by straight-line segments. However, it should be understood that a plurality of points can be connected by curved lines with predetermined curvatures. Preferably, the profile of a spacer of an intervertebral implant can be curved by polynomial interpolation, spline interpolation, and others. In particular, in the case of using spline interpolation, a plurality of points can be smoothly connected by polynomial interpolation. Furthermore, spline interpolation produces a smooth curving by connecting line segments using a quadratic function, a cubic function, a quartic or higher function according to slope intervals.

Although the above-described embodiment has been described in terms of a first notch 33a, it can be applied to a second notch 33b.

Hitherto, clustering for the profile slope of a first notch 33a from various patient groups has been described. In addition, to perform a common clustering considering various factors such as the shape, size, and a spacing of the flanges, a spinal image clustering method using a spinal image case can be considered. Although the size and shape of spinous processes may vary according to sex and individual differences, the number of intervertebral implants to be substantially manufactured must be restricted. For this, the most common spinous process shapes and sizes must be known. Hereinafter, a method of finding the most common shapes according to the number assigned by a user will be described.

Generally, clustering analysis can be used to reduce the data and to categorize similar data. Such clustering has been widely applied in information processing. An object of the clustering algorithm is to provide an automated tool for performing or assisting categorization and classification. Clustering does not require discrimination between independent variables and dependent variables and previous classification of data sets. The purpose of clustering is to find similar groups in expectation that similar records will behave similarly.

Thus, the most important consideration in data comparison and integration is to reduce the size of data sets while not losing the inherent characteristics of the data. It is important to select a method that ensures efficient computation and that can find data sets capable of representing the original data. Data set finding is generally based on computation of a mean value of the measured values. In the present invention, there is provided an improved k-means clustering algorithm as a method of finding the shape distribution of spinous processes.

Cluster Determination

FIG. 29 is a schematic block diagram illustrating a spinal image clustering system according to an embodiment of the present invention. Referring to FIG. 29, the spinal image clustering system includes a user interface 110, a class assignment control module 120, a control module 130, an original image database 140, a representative image database 150, a binarized image database 160, an image collection module 170, a volume of interest (VOI) extraction module 180, and a VOI binarization control module 190.

The user interface 110 receives a user-selected VOI among a full-field spinal image or it manages input/output for image collection.

The class assignment control module 120 assigns cases to existing classes according to a similarity threshold determined by a learning module or it creates new classes.

The control module 130 mainly serves to correct a representative image when new cases are contained in existing classes. In addition, the control module 130 compares a representative image of a previous round and a representative image of a present round for convergence, which leads to re-clustering by increasing the similarity threshold when the number of assigned classes is less than a predetermined number.

The original image database 140 stores images collected by the image collection module 170 without correction, and provides original images when binarized images are created or when representative class images are corrected.

The representative image database 150 stores representative images reflecting the characteristics of classes. Here, the representative images are mean images of original images belonging to classes.

The binarized image database 160 stores binarized VOI images extracted from original images. Binarized images of the representative images are also stored in the binarized image database 160. The binarized image database 160 is used in comparing the binarized images of cases with the binarized images of the representative images to assign cases to corresponding classes.

The image collection module 170 receives original images from an external source, and then stores the received original images in the original image database 140 or it transmits the received original images to the VOI extraction module 150.

The VOI extraction module 180 receives original images from the image collection module 170. When the VOI is extracted by a user, the VOI extraction module 180 transmits the extracted VOI to the VOI binarization control module 190.

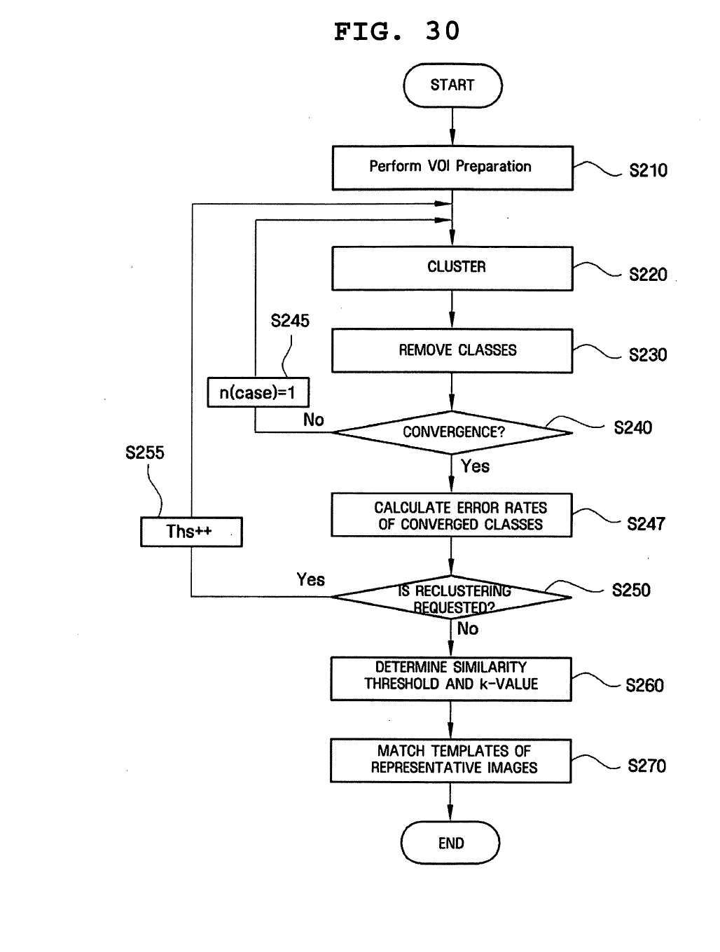

The VOI binarization control module 190 receives the VOI from the VOI extraction module 170, binarizes the received VOI according to a predetermined standard, and transmits the binarized VOI to the binarized image database 160. Automated image classification and management using the spinal image clustering system of the present invention will now be described in detail with reference to FIGS. 30 through 41.

FIG. 30 is a flow diagram illustrating spinal image clustering according to an embodiment of the present invention. Referring to FIG. 30, VOI preparation is first performed prior to clustering. That is, the VOI is extracted and binarized, and a first case is assigned to a first class (hereinafter, referred to as “class A”) S210.

Image matching is performed for a binarized VOI image of a second case and a binarized representative image of class A. When the binarized VOI image of the second case is matched to the binarized representative image of class A within predetermined limits, the second case is contained in class A and a representative image of class A is corrected. On the other hand, when the binarized VOI image of the second case is not matched to the binarized representative image of class A, the second case is assigned to a newly created class B. This procedure is iterated for all cases. That is, when the binarized VOI of an n-th case is compared with the binarized representative images of existing classes A through P, the n-th case is assigned to its matched class. If there are no binarized representative images matching the binarized VOI of the n-th case, the n-th case is assigned to a newly created class P+1. When the assignment for the last case is terminated, classes containing less than a certain ratio (e.g., less than 2%) of cases (based on total cases) are removed (operation S230). The class removal may be selectively performed for the purpose of separately extracting cases with exceptional and different shapes.

After exceptional classes are removed, whether correction of the representative image of each class has been made is investigated. If the correction of a representative image has been made, clustering and class removal are again performed; however, if convergence is achieved, the number of classes is determined. The above procedure is iterated until all the representative images have been corrected (operation S240).

Next, the error rates of converged classes are calculated (operation S247). If the number of classes determined using the similarity threshold is smaller than the maximum number of permissible classes, the clustering operation, the class removal operation, and the convergence determination operation are iterated using an increased similarity threshold S250.

Additionally, matching templates of representative images completed according to the above process are matched to the cases to select more common representative images S260.

Cluster Determination Procedure

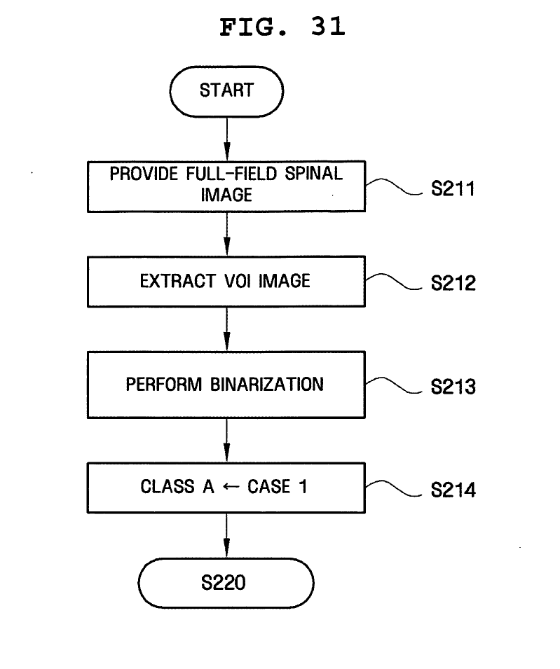

Hereinafter, each operation of FIG. 30 will be described in more detail with reference to the accompanying drawings. FIG. 31 is a detailed flow diagram of the preparing operation S210 of FIG. 30. The preparing operation includes selection of a VOI image from a full-field spinal image and binarization of the VOI image.

As shown in FIG. 32, when the full-field spinal image with 256 gray levels is provided (sub-operation S211), a VOI image is extracted by a user. In this embodiment, the VOI is represented by a three-dimensional cuboid using xyz-coordinates. First, image slices of the implant area are selected and regions of interest (ROIs) are defined in the slices. The VOI is defined by adding an extra volume to the location of an installed intervertebral implant, and several slices are selected in the upper and lower z-axis. All images of the ROIs are the same resolution. For example, 10 slices may be selected in the z-direction, and all images may consist of 64*64 pixels. Furthermore, in one case, ROIs may be the same in all slices. These cases are represented as: {S₁, S₂, S_{N}}.

Then, binarization is performed using Equation 2 below (sub-operation S213). All cases are grouped into k clusters {P^{t}ⱼ, 1≦j≦k}. If a case belongs to cluster Pⱼ, the case is classified into representative image vectors _{c}μ^{t}ⱼ (j=1, k). Here, t is a round and c is the number of updates atj. B(_{c}μ^{t}ⱼ(x,y,z)) is a function for binarization of the gray level of each voxel at the (x,y,z) position of the representative image vector.

$$B ( {}_{c}^{}{μ}{}_{j}^{t} ( x , y , z ) ) \{ \begin{matrix}0 & if & {}_{c}^{}{μ}{}_{j}^{t} ( x , y , z ) < 230 \\ 255 & if & {}_{c}^{}{μ}{}_{j}^{t} ( x , y , z ) \geq 230\end{matrix} \qquad ( 2 )$$

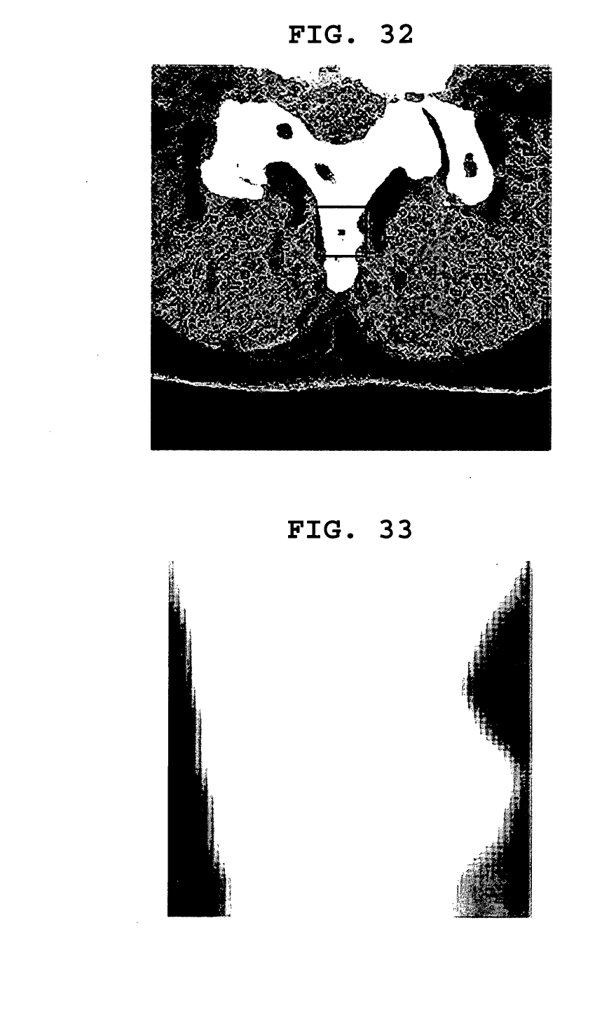

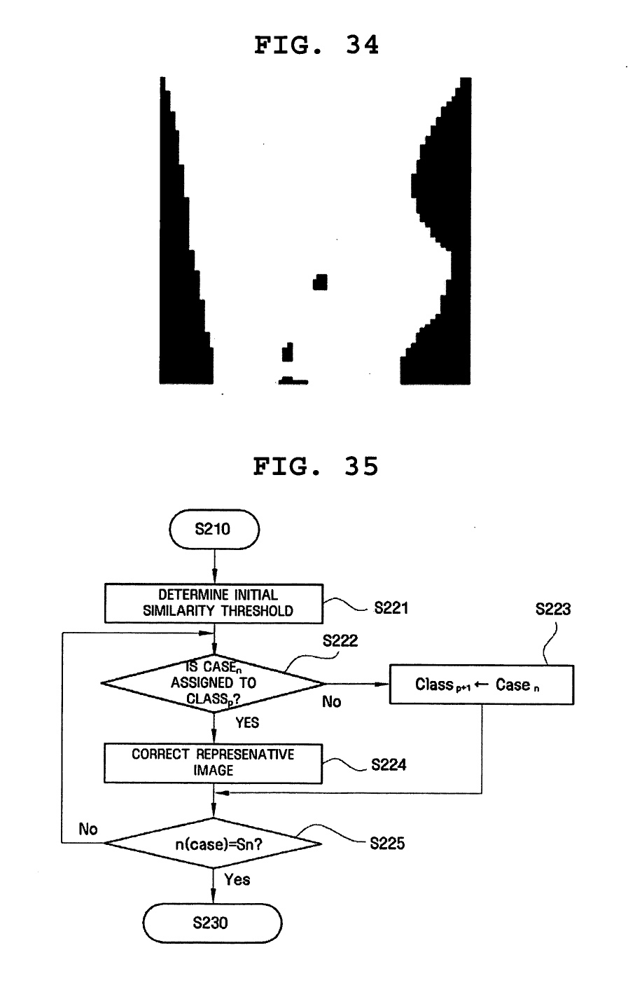

A threshold value for spinal image binarization may be optionally selected from the range 190 to 240. However, considering minimal noise and spinal density, 230 is preferable. For persons with a low bone mineral density, 200 may be selected as the threshold value for spinal image binarization. The threshold value for spinal image binarization is not limited to the above-described values. FIG. 33 shows a ROI image with 64*64 pixels and FIG. 34 shows a binarized image of the ROI image of FIG. 33.

The preparing operation is terminated by assigning a first case to a class A (sub-operation S214).

FIG. 35 is a detailed flow diagram of the clustering operation S220 of FIG. 30.

Referring to FIG. 35, first, when t=0, an initial similarity threshold (Thₛ=Thₛₜₐᵣₜ) is determined for class assignment (sub-operation S221). The initial similarity threshold may be directly determined by a user or it may be randomly determined in a predetermined range. For example, the initial similarity threshold may be selected from a range of 70 to 100%. In this embodiment, 76% is selected as the initial similarity threshold. However, since clustering results are not significantly affected by the initial similarity threshold, an appropriate value should be selected as the initial similarity threshold.

Then, an n-th case is assigned to a p-th class (sub-operation S222). According to Equation 3 (below), a binarized gray level of a case to be processed is compared with a binarized gray level of a representative image of a class of a given voxel position and a matching rate of the two binarized gray levels is calculated. That is, the cumulative value of pixels in the matched areas of the binarized gray level of a case and the binarized gray level of the representative image of a class is represented by a percentage. In Equation 3, A_{case} is the number of pixels in the VOI, i.e., the number of pixels in ROI*the number of slices. In this embodiment, A_{case} is 64*64*10. Con[Sᵢ, _{c}μ^{t}ⱼ] is a function for calculating a matching rate of gray levels when a comparison between a binarized image of a case and a binarized image of a representative image of a class is iterated A_{case} times.

$$Con [ {S}_{i} \text{:} {}_{ c}^{}{μ}{}_{j}^{t} ] ( \% ) = \frac{{\sum }_{1}^{{A}_{case}} \{ {S}_{i} ( x , y , z ) / B ( {S}_{i} ( x , y , z ) = B ( {}_{c}^{}{μ}{}_{j}^{t} ( x , y , z ) \}}{{A}_{case}} * 100 ( \% ) \qquad ( 3 )$$

In the matching rate calculation method, gray levels and position information are compared at the same time. Therefore, the matching rate of gray levels of fill-field volumes can be calculated by comparing the gray levels of corresponding voxels.

After performing matching, Con[Sᵢ, _{c}μ^{t}ⱼ] is compared with the previously determined initial similarity threshold. If Con[Sᵢ, _{c}μ^{t}ⱼ] is less than the initial similarity threshold, the n-th case is assigned to a newly created p+1-th class (sub-operation S223). If Con[Sᵢ, _{c}μ^{t}ⱼ] exceeds the initial similarity threshold, a representative image is corrected (sub-operation S224).

In more detail, in assignment of a case to a class, a first case S₁ is assigned to a class 1. With respect to a second case S₂, a gray level of a representative image of the class 1 assigned as cluster 1 is compared with that of a corresponding voxel of the second case S₂. If a matching rate of the two gray levels is less than an initial similarity threshold, the second case S₂ is assigned to a cluster 2. On the other hand, if it exceeds the initial similarity threshold, the second case S₂ is assigned to the class 1 and the representative image of the class 1 is corrected. With respect to i-th case Sᵢ (i is more than 2), the gray level of the binarized representative image of the previously created j-th class Pⱼ (j=1, . . . , k) is compared to that of its corresponding voxel of the i-th case Sᵢ using Equation 4 (below). The i-th case Sᵢ is assigned to the j-th class Pⱼ exhibiting the highest matching rate. Representative image correction is required in the j-th class Pⱼ to which the i-th case Sᵢ is assigned.
Sᵢε{pⱼⁱ/MAX(Con.[Sᵢ:μⱼ^{t}])}, (j=1, 2, . . . k) (4)

In the sub-operation S224 (correcting a representative image), the gray level of a pixel is corrected using 256 gray levels instead of a binarized image. If the number of cases belonging to the j-th class Pⱼ before correction is c, a new representative image is represented by Equation 5 (below). A procedure of correcting a representative image using Equation 5 is defined as a “learning procedure”, and the ratio of the new _{c+1}μ^{t}ⱼ to the previous _{c}μ^{t}ⱼ is defined as the “learning rate”.

$${}_{c + 1}^{}{μ}{}_{j}^{t} = ( \frac{c}{c + 1} ) * {}_{c}^{}{μ}{}_{j}^{t} + ( \frac{1}{c + 1} ) * {S}_{i} \qquad ( 5 )$$

The new representative image represented by Equation 5 is determined on the basis of averaging existing cases and a newly assigned case. According to such a representative image determination method, unlike a representative image determination method considering a simple mean value of existing cases belonging to the same class as a representative image vector, a variable representative image vector that may be changed by new case assignment is obtained, thereby ensuring accurate clustering and representative image acquisition.

The learning rate is inversely proportional to the number of cases assigned to a corresponding class. In the case of processing one training case, since a representative image value of a class to which the training case belongs is previously determined by c existing cases, the learning rate of a single case decreases as the number of existing cases determining a representative image increases.

The reason the learning rate decreases is that a representative image is determined by a cumulative value of all cases assigned to a corresponding class. A representative image of each class is considered a representative image of all cases assigned to each class. Thus, classes are discriminated only by such representative images, and each case is assigned to a class having the closest representative image.

After a representative image is corrected, sub-operations S222 through S224 are iterated until the assignment of all cases is completed, i.e., until the last case S_{N} is processed (sub-operation S225). When the assignment of the last case is terminated, classes containing less than 2% of cases (based on total cases) are removed from a class list using Equation 6 (operation S230). Since exceptional classes are removed, k significant classes remain. Therefore, disadvantages of an algorithm sensitive to a k-value and an exceptional value can be solved. Thus, operation 230 can be intended for separate extraction of cases with exceptional and specific shapes.

$${p}_{remove} = \{ {p}_{j}^{t} / \sum {S}_{m} < ( {\sum }_{1}^{N} {S}_{i} * \frac{2}{100} ( \% ) ) , {S}_{m} ∈ {p}_{j}^{t} \} \qquad ( 6 )$$

Table 5 presents the results for a class removal of 100 cases. With respect to L34_upper at a similarity threshold of 76%, four classes are created and classes composed of 2 or smaller cases are removed. As a result, a total of four cases are removed.

TABLE 5

| | Section | | | |
|---|---|---|---|---|
| | L34_upper | | L34_lower | |
| Similarity | | The number of | | The number of |
| threshold | k-value | removed cases | k-value | removed cases |
| 76 | 4 | 4 | 2 | 2 |
| 77 | 4 | 3 | 2 | 3 |
| 78 | 4 | 4 | 2 | 6 |
| 79 | 4 | 4 | 2 | 6 |
| 80 | 5 | 5 | 2 | 6 |
| 81 | 6 | 3 | 3 | 6 |

When the class removal is terminated, the convergence determination operation S240 for stabilization of each class is executed.

In the convergence determination operation, whether correction of the representative image of each class has been made is investigated. Rounds are iterated until the convergence of the representative images is accomplished. Here, “rounds” refers to iteration of clustering and class removal. In this regard, an “iteration of 3 rounds” means that clustering and class removal are iterated three times.

As shown in Equation 7, rounds are iterated until the matching rate of the representative image of each class of a previous round and the representative image of a newly created class exceeds a predetermined percent. The matching rate may be optionally determined by a user. For example, rounds may be iterated until the matching rate is 100%.
Con[ₗμ^{t}ⱼ:ₗμ^{t-1}ⱼ](%)=100(%) (7)

If the matching rate does not exceed a predetermined percent, it is considered that convergence has not been accomplished. In this case, case assignment must be done again. Thus, a case number is initialized to “1” (operation S245) and then rounds are iterated. If convergence is accomplished, the operation S247 of calculating an error rate is executed.

Here, the error rate of converged classes is calculated S247. As shown in Equation 8, the error rate is defined as the sum of the squares of the distance between each case in a class and the representative image of the class. The “distance” is conceptual. Actually, the error rate is represented by an error observation calculated on the assumption that dissimilarity between each case and its representative image is 100%. After calculating the error rate based on the distance between each case and its representative image, the re-clustering determination operation S250 is executed.

$$E = {\sum }_{j = 1}^{k} {\sum }_{Si} {d}^{2} ( {S}_{i} , {μ}_{j} ) \qquad ( 8 )$$

In the re-clustering determination operation, when no representative image correction is done and the number of classes is smaller than the number of permissible classes, the operation S255 of increasing the similarity threshold using Equation 9 is executed. If re-clustering is requested, clustering is again executed using an increased similarity threshold S255. If re-clustering is not requested, the similarity threshold and the k-value are determined S260. Since the initial similarity threshold (Thₛₜₐᵣₜ) is an approximate value, the k-value determined from the initial similarity threshold is also not completely suitable for assignment of given cases. Thus, through the above process, the maximum similarity threshold (Thₛ) can be obtained within permissible classes. For example, if the number of classes is smaller than the number of permissible classes, the process returns to the operation of clustering S220 and increases the similarity threshold by 1% to iterate assignment of all cases to classes.

When

$$J = {\sum }_{1}^{j} {p}_{j}^{t} and J 〈 {K}_{L} , {th}_{S} ++ \qquad ( 9 )$$

In the operation to determine the similarity threshold and k-value S260, the similarity threshold and k-value corresponding to the smallest error rate among error rates calculated with an increased similarity threshold are determined to be the most suitable. The squares of the distance according to the similarity threshold, and error rates calculated after excluding cases removed in the class removal operation are listed in Table 6 below. As shown in Table 6, the smallest error rate, 67414, was obtained at the similarity threshold of 82%. Thus, it can be seen that it is most preferable that the initial similarity threshold be 82%.

TABLE 6

| Similarity | Square of | The number | |
|---|---|---|---|
| threshold (Th) | distance (d) | of error cases | Error rate |
| 80 | 9312 | 6 | 69312 |
| 81 | 7800 | 6 | 67800 |
| 82 | 7414 | 6 | 67414 |
| 83 | 7293 | 7 | 77293 |
| 84 | 7226 | 9 | 97226 |
| 85 | 6662 | 7 | 76662 |

After the similarity threshold and the k-value are determined, the representative image matching operation S270 (FIG. 30) may be selectively performed. When all cases are scanned using intervertebral implants suitable for the representative images of classes, the number of classes can be reduced and a universal representative image can be created. When an intervertebral implant is not fitted with spinous processes at a specific spot during scanning, the scanning is stopped and then again performed at another spot. During the scanning, the minimal error spot, i.e., the minimal error space spot is searched and recorded. This matching procedure is performed by a voxel-based comparison of cases 271 and templates 273 configured for three-dimensional intervertebral implants. For example, the full-field of a case has a volume of 64 (length)*64 (width)*10 (the number of slices), and a template has a volume of 48*48*4. The full-filed is searched according as the template shifting per 1 voxel with respect to the case. In this case, a variable interval (x, y, z)=(17, 17, 5).

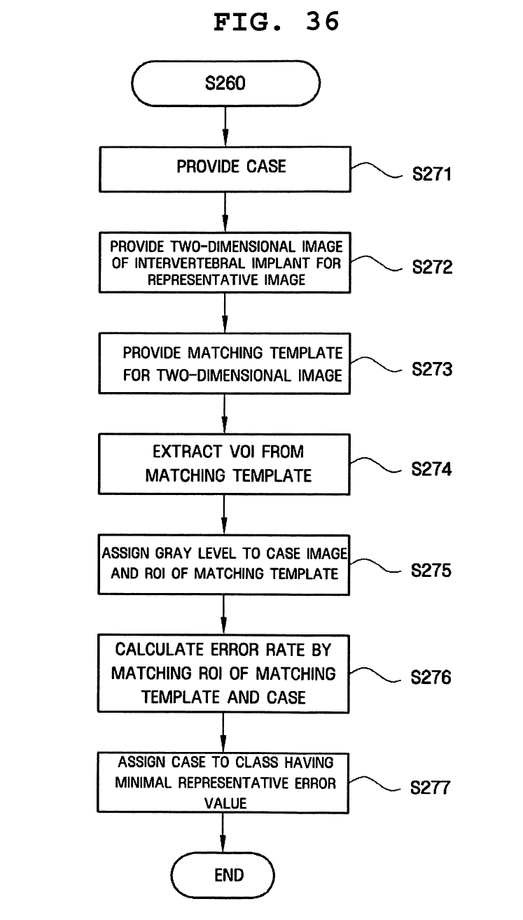

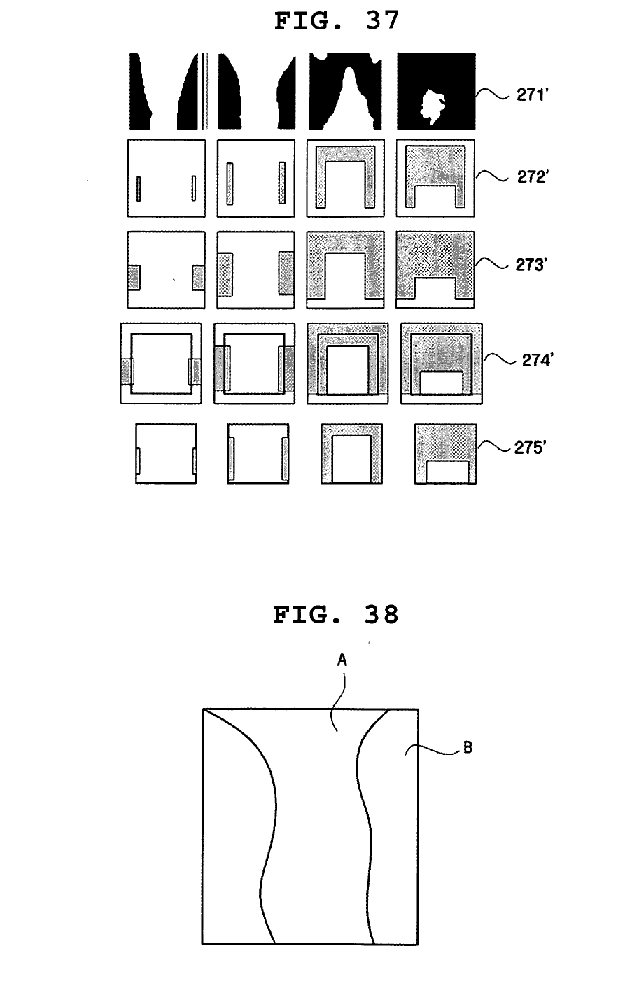

FIG. 36 is a detailed flow diagram of the representative image matching operation S270 of FIG. 30 and FIG. 37 is an exemplified view of the representative image matching. The representative image matching can be efficiently used in manufacturing a universal intervertebral implant suitable for most cases.

In addition, the purposes of the representative image matching can be divided into two groups. First, an intervertebral implant has geometrically curved surfaces, and thus may differ from the three-dimensional shape of the representative image. The representative image matching reduces the number of intervertebral implants by a quantitative measurement of the error space between an intervertebral implant and a case, and ensures intervertebral implants with better adapted shapes. Second, since an error rate between a representative image and a single case equals the similarity threshold, an intervertebral implant is manufactured to large dimensions considering the error rate. The representative image matching is to verify the enlarged dimensions.

After determining the similarity threshold and the k-value, a case is provided (sub-operation S271). The case is provided in a comparable shape. In this embodiment, there is provided a case having a volume of 64*64*10.

Then, a two-dimensional image of an intervertebral implant adapted for a representative image is provided (sub-operation S272). First, in the representative image, the widths of the spinous processes, an interspinous space, lower surface slopes of the spinous processes, the depths of the spinous processes, and others are measured. Then, an intervertebral implant is manufactured so that it has slightly larger dimensions than the representative image. The intervertebral implant is now designated a “representative pattern.” Furthermore, a plurality of representative patterns can be manufactured according to the degree of margin in the dimensions. Adaptability of the representative patterns is determined through a matching procedure.

The two-dimensional image of the intervertebral implant is provided so that a matching template can be provided and a region of interest (ROI) can be extracted from the matching template. For this, the two-dimensional image of the intervertebral implant may be provided in the same form as the case. Preferably, the intervertebral implant is provided as a cubic image of 64*64*10.

Then, a matching template adapted for the two-dimensional image of the intervertebral implant is provided (sub-operation S273). This sub-operation is to detect the presence of spinous processes in a 180-degree range based on the centroid of the lower portion of the representative pattern both inside and outside the template. The matching template has a volume of 48*48*4. Referring to FIG. 37, a matching template 273 is slightly larger than the two-dimensional image of the representative pattern to perform the matching with a case image.

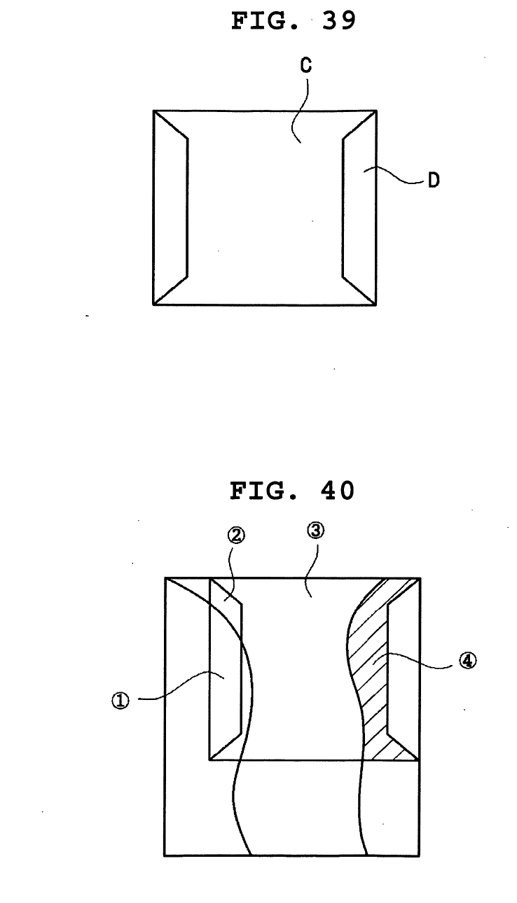

Then, a ROI is extracted from the matching template (sub-operation S274). Referring to FIG. 37, a portion of the matching template is selected as a ROI 274. For example, in the case of using a matching template with a size of 64*64, a ROI with a size of 48*48 may be defined. In this case, the 48*48 ROI can scan a case region of 64*64 along with a variable space of 17*17. When the ROI is defined, a gray level is assigned to the case image and the ROI of the matching template to discriminate the ROI from the case image, which is a target of comparison (sub-operation S275). Referring to FIGS. 38 and 39, in a case image, zero (0) is assigned to a spinous process A and 255 is assigned to a background B. In a ROI of a matching template, 100 is assigned to a background C and 255 is assigned to a matching template D. Since gray level assignment can also be performed using another gray level capable of discriminating the four regions, the present invention is not limited to the above-described examples.

After the gray level assignment, an error rate is calculated by matching the ROI of the matching template and the case (sub-operation S276). Analysis results for the matching are given below with reference to FIG. 40.

Referring to FIG. 40, there are about four regions. A region 1 containing only a template has a gray level of (255+255)/2 and is skipped in processing. A region 2 is an overlap of the case and the template and has a gray level of (0+255)/2. In the region 2, because the spinous process of the case is larger than an intervertebral implant, there exists a protrusion of the spinous process from the template. When a voxel is detected during processing, the intervertebral implant is considered to be inappropriate. A region 3 contains only the spinous process of the case and has a gray level of (0+100)/2. Like region 1, region 3 is also skipped since it does not affect the processing. A region 4 contains only the backgrounds of the two images and represents an error space between the template and the case. Region 4 has a gray level of (255+100)/2, and the number of voxels is added cumulatively. The cumulative value of region 4 is computed as an error value and it is used to calculate the similarity threshold. The analysis results are summarized in Table 7:

TABLE 7

| Region | | | | | |
|---|---|---|---|---|---|
| No. | Section | Case | Template | Gray level | Processing |
| 1 | Template | — | ◯ | (255 + 255)/2 | Skipped |
| 2 | Spinous | ◯ | ◯ | (0 + 255)/2 | Inappropriate |
| | process + | | | | |
| | template | | | | |
| 3 | Spinous | ◯ | — | (0 + 100)/2 | Skipped |
| | process | | | | |
| 4 | Background | — | — | (255 + 100)/2 | Error value |
| | | | | | calculation |

In region 4, the spinous process is not matched to the intervertebral implant. As the error value increases, a space between the intervertebral implant and the spinous process increases. Thus, it is important to reduce the error value. While scanning the matching template with respect to the full-field image of the case along the variable space, cumulative error values are calculated based on all error calculations. That is, in the case of matching a single matching template with a size of 48*48*4 and four slices, cumulative error values for the slices are calculated.

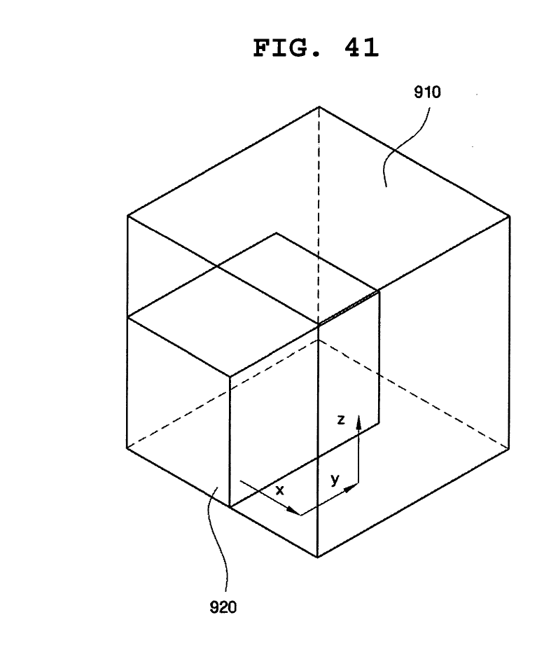

Referring to FIG. 41, a matching template 920 is scanned along a variable space (17*17*5) in the x, y, or z direction in a case 910, and a cumulative error value for each position of the variable space is calculated. By doing so, even when a matching template image and a case image are not accurately registered in terms of length, width, height, and angle, the position of the matching template can be detected.

For example, the above-described matching and error rate calculation can be implemented using Visual C++ as follows.

For(Z_range){

For(X_range){

For(Y_range){

if(region_1)

;

else if(region_2)

loop exit;

else if(region_3)

;

else

error_rate++;

}

}

}

If(error_rate > threshold)

exit;

x_range, y_range, and z_range are iterated by the initial For. If regions 1 and 3 are present, processing is skipped; if region 2 is present, a loop exit occurs; and if region 4 is present, an error value is cumulatively calculated.

When 17*17*5 cumulative error values are computed through this process, the minimal cumulative error value is defined as the representative error value of the case for the intervertebral implant of the representative image.

The scanning and the representative error value determination must be iterated for each representative image and each case. The iteration of scans can be calculated by Equation 10:
Iteration=x_range*y_range*z_range*k*case (10)
where x_range, y_range, and z_range are respectively x, y, and z values of the variable space, k is the predetermined number of classes or representative images, and case is the number of cases. According to Equation 10, scanning for a single representative image and a single case with a variable space of 17*17*5 is iterated 1,445 (17*17*5*1*1) times. Scanning for 40 representative images and 100 cases is iterated 5,780,000 (17*17*5*40*100) times.

When the representative error values of a case for an intervertebral implant corresponding to the representative image of each class are calculated through the iteration, the case is assigned to the class having the minimal representative error value (sub-operation S277). As shown in Table 8 below, a case 1 is assigned to a class B since it has the minimal representative error value in the class B. Case assignment to class may also be implemented using the representative error rate indicating the ratio of each representative error value to the total of representative error values. In this case, a case is assigned to a class having the minimal representative error rate. By calculating (100-representative error rate) %, a case may also be assigned to a class having the maximal (100-representative error rate) %. Thus, case assignment is not restricted by parameters.

TABLE 8

| | Intervertebral implant | | |
|---|---|---|---|
| | corresponding to | Representative | |
| Case | representative image | error value | Assignment |
| Case 1 | Class A | 69,300 | Class B |
| | Class B | 67,800 | |
| | Class C | Mismatch | |
| | Class J | 76,666 | |

An intervertebral implant is manufactured with a margin of error, and therefore it does not perfectly fit an interspinous space. Thus, the same intervertebral implant can be used for the representative image of another class. Furthermore, because of a minute image difference due to a high k-value and similarity threshold, case assignment to another class can be implemented. In this regard, the representative image matching produces more general intervertebral implants. However, when production of various types of intervertebral implants is desired, the representative image matching may be omitted.

Table 9 presents (100-representative error rate) % of each case in each class. As described above, a representative error value or a representative error rate may be used instead of (100-representative error rate) %. Table 9 shows matching results between each case and an intervertebral implant corresponding to the representative image of each class. From the matching results, a matching rate can be estimated. Also, since classes with very low matching rates can be filtered out, the matching results of Table 9 can be used as primary evaluation standards.

TABLE 9

| Section | Class A | Class B | Class D | Class F | Class H |
|---|---|---|---|---|---|
| Case 1 | 86.4 | 81.2 | 72.7 | 80.6 | 69.7 |
| Case 2 | 0 | 81.9 | 79.4 | 0 | 82.1 |
| Case 3 | 0 | 0 | 79.9 | 0 | 77.6 |
| Case 4 | 0 | 0 | 79.5 | 0 | 77.2 |
| Case 5 | 0 | 76.9 | 72.8 | 81.1 | 66.9 |
| Case 6 | 0 | 88.1 | 76.7 | 0 | 78.8 |
| Case 7 | 88.5 | 90 | 82.9 | 0 | 78.5 |
| Case 8 | 0 | 90 | 82.9 | 0 | 78.5 |
| Case 9 | 77.2 | 61.5 | 51.9 | 59.9 | 47.5 |
| Case 10 | 0 | 92.8 | 85.9 | 0 | 82.5 |
| Case 11 | 0 | 84 | 73.6 | 0 | 71.3 |
| Case 12 | 0 | 89.3 | 83.1 | 0 | 77.9 |
| Case 13 | 83 | 78.5 | 68.4 | 0 | 65.4 |
| Case 14 | 0 | 81.5 | 66.2 | 0 | 68.7 |
| Case 15 | 0 | 83.8 | 68.8 | 0 | 73.4 |
| Case 16 | 0 | 0 | 77.7 | 0 | 71.7 |
| Case 17 | 0 | 93.1 | 87.1 | 0 | 86.9 |
| Case 95 | 0 | 88.2 | 76.5 | 0 | 80.6 |

Table 10 presents a mean matching rate for each class, the number of assigned cases, and an optimal determination rate. The mean matching rate indicates a mean of the (100-representative error rate) % values shown in Table 9, the number of cases indicates the number of cases assigned to each class, and the optimal determination rate indicates the ratio (%) of cases assigned to each class to the total number of cases. For example, the optimal determination rate of class A is (19/95)*100=20%.

TABLE 10

| Section | Class A | Class B | Class D | Class F | Class H |
|---|---|---|---|---|---|
| Mean matching rate | 13.94 | 75.99 | 72.79 | 12.51 | 74.84 |
| for each class (%) | | | | | |
| The number of cases | 19 | 60 | 4 | 1 | 11 |
| Optimal determination | 20 | 63 | 4 | 1 | 12 |
| rate (%) | | | | | |

According to the above-described embodiment, a spacer is made of solid material and ensures a predetermined space between two spinous processes. After surgery, spinal motion may occur. In this case, if a spacer is perfectly fixed between spinous processes, patient movement may be restricted and other normal vertebrae may be adversely affected. Thus, there is need to design a spacer having elasticity so that a flexible space between upper and lower parts of the spacer (within a limited range) can accommodate spinal motion.

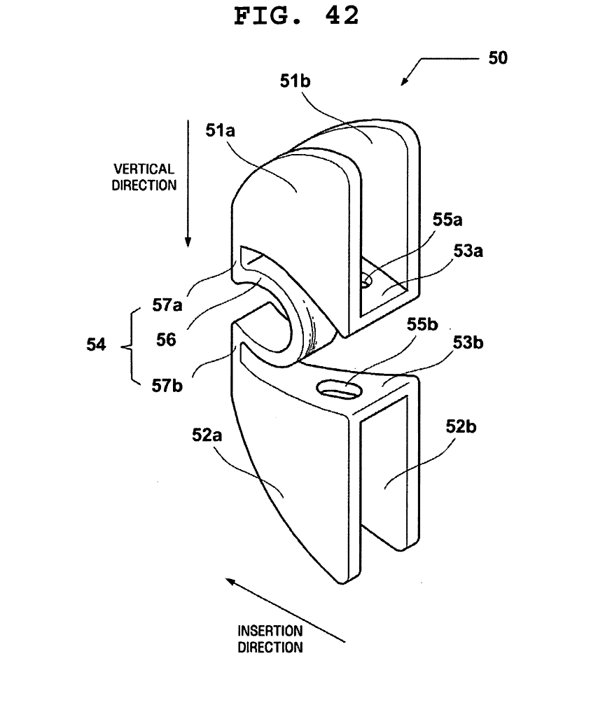

In view of the above problem, several spacers according to other embodiments of the present invention will be described hereinafter. FIG. 42 is a perspective view illustrating a spacer 50 according to another embodiment of the present invention.

Referring to FIG. 42, the spacer 50 includes flanges 51a, 51b, 52a, and 52b and first and second notches 53a and 53b, like the above-described spacer 30. However, the spacer 50 slightly differs from the spacer 30 in that an elastic folding portion 54 imparting elasticity to the spacer 50 is further included and the first and second notches 53a and 53b are respectively formed with through-holes 55a and 55b.

The elastic folding portion 54 is positioned at an insertion front part, and has one or more folds to impart elasticity to the spacer 50. Due to such a structural feature, even when the spacer 50 is made of a solid metal such as titanium, the space between upper and lower parts of the spacer 50 can be elastically changed within a predetermined range. In more detail, the elastic folding portion 54 may include a vertical straight line portion 57a connected to the first notch 53a, a vertical straight line portion 57b connected to the second notch 53b, and a curved portion 56 connecting the two straight line portions.

The straight line portions 57a and 57b serve to ensure a space corresponding to at least the sum of heights of the straight line portions 57a and 57b between spinous processes to prevent excessive spinal extension. The curved portion 56 serves to ensure elasticity of the elastic folding portion 54 upon extension or flexion.

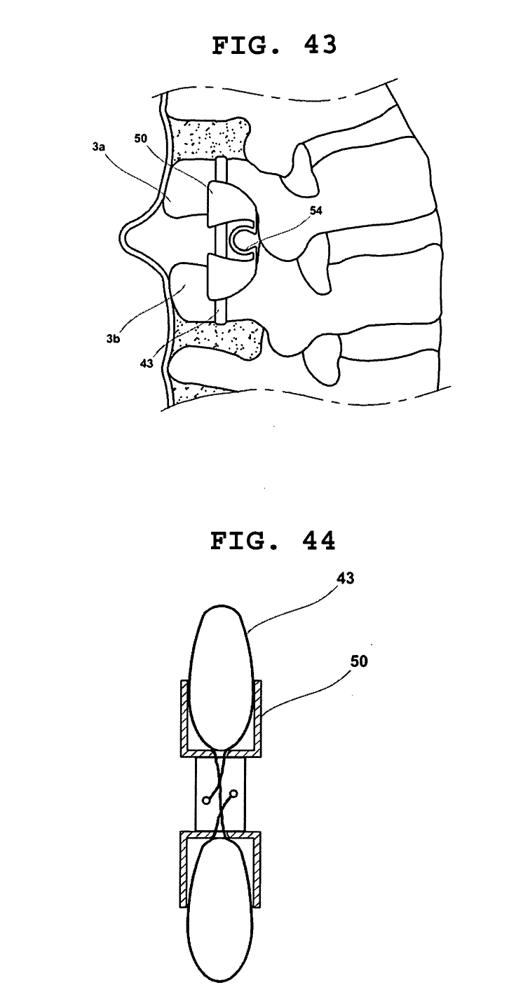

FIG. 43 is a diagrammatic lateral view of a spinal column illustrating the placement of the spacer 50 of FIG. 42 between adjacent spinous processes. Referring to FIG. 43, a band 43 is inserted into the through-holes 55a and 55b of the spacer 50, as in the embodiment of FIG. 3. The band 43 binds an upper spinous process 3a, a lower spinous process 3b, and the spacer 50. A sectional view of the spacer 50 bound with the band 43 is shown in FIG. 44. The band 43 surrounds the upper spinous process 3a and the lower spinous process 3b one or more times in a FIG. 8. Then, end portions of the band 43 are fixedly knotted.

To prevent contact between the elastic folding portion 54 and the band 43 when the band 43 is inserted into the through-holes 55a and 55b, it is preferable that the band 43 be separated from the elastic folding portion 54 by a predetermined distance in the left and right direction of FIG. 43.

When the spacer 50 is placed between two adjacent spinous processes 3a and 3b as shown in FIG. 43, the space between the spinous processes 3a and 3b may be elastically changed. Therefore, the spacer 50 can adapt to a patient's motion while maintaining the space between the spinous processes 3a and 3b. That is, abnormal spinal motion can be restricted while normal spinal motion can be permitted. Furthermore, since opposing notches of the spacer 50 indirectly contact the spinous processes 3a and 3b via the band 43, external impacts can be buffered.

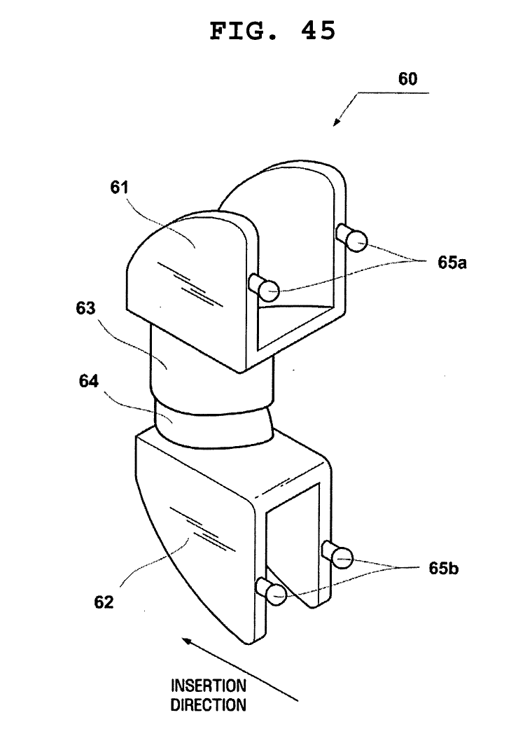

Meanwhile, a spacer 60 according to a still another embodiment of the present invention is shown in FIG. 45. Referring to FIG. 45, the spacer 60 is divided into upper and lower bodies 61 and 62 with notches.

A lower portion of the upper body 61 is formed with a cylindrical receiver 63 and an upper portion of the lower body 62 is formed with an insertion member 64. The insertion member 64 is partially inserted into the cylindrical receiver 63. A separate connector is not provided for the coupling between the insertion member 64 and the cylindrical receiver 63.

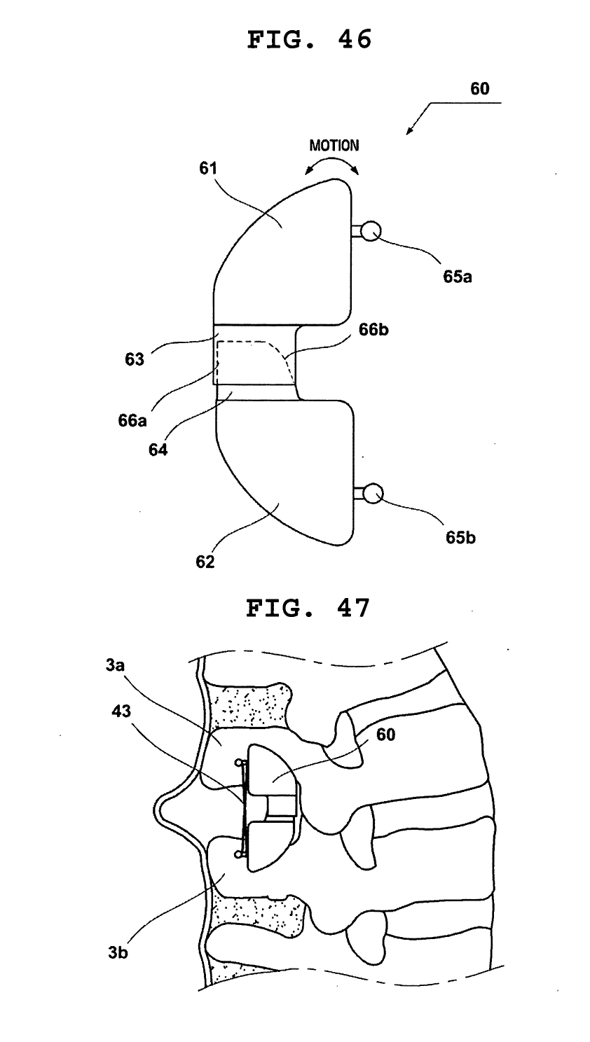

The coupling between the insertion member 64 and the cylindrical receiver 63 will now be described in more detail with reference to FIG. 46. The cylindrical receiver 63 has a simple cylindrical shape. The insertion member 64 has such a shape that it can be partially inserted into the cylindrical receiver 63. The insertion member 64 has a conic shape that a first angle portion 66a of the left side (insertion front part) is steeper than a second angle portion 66b of the right side (insertion rear part). Since there is no separate connector connecting the cylindrical receiver 63 and the insertion member 64, it is preferable that the first angle portion 66a and the second angle portion 66b have a curved shape to ensure relative movement of the cylindrical receiver 63 and the insertion member 64.

When the first angle portion 66a is steeper than the second angle portion 66b, a right-directed rotation of the cylindrical receiver 63 with respect to the insertion member 64 is retarded and a left-directed rotation is facilitated. That is, excessive extension can be prevented, and slight flexibility can be ensured.

Returning to FIG. 45, the upper body 61 and the lower body 62 are respectively formed with band fixing projections 65a and 65b to bind the fixing projections 65a and 65b with the band (43 of FIG. 43). The band 43 allows slight flexion within an elastic range due to the gradual slope of the second angle portion 66b, but it does not allow flexibility for extension due the steep slope of the first angle portion 66a. Because of the slight elasticity of the band 43, the notches can naturally contact the spinous processes during lordosis.

FIG. 47 is a diagrammatic lateral view of a spinal column illustrating the placement of the spacer 60 of FIG. 45 between adjacent spinous processes. Referring to FIGS. 45 through 47, the band fixing projections 65a and 65b are bound with a band 43. The tension of the band 43 can be maintained by contacting the first angle portion 66a of the insertion member 64 with an inner surface of the cylindrical receiver 63. Upper and lower spinous processes 3a and 3b may also be bound with a separate band near the pivot of the spacer 60.

As described above, the spacer 60 has separate bodies independently supporting spinous processes. Therefore, the motion of spinous processes can be maximally permitted. Also, since a separate force is present, the natural lever system of the human spine can be ensured.

In the embodiments of FIGS. 42 and 45, the notches have a profile shape perpendicular to the flanges 44. However, those of ordinary skill in the art can appreciate that the profile shape of a notch may be conformally curved to fit with a lower surface of a spinous process.

In addition, the profile shapes of the notches of the embodiments shown in FIGS. 42 and 45 can be determined by the clustering method described with reference to FIGS. 22 through 28.

### INDUSTRIAL APPLICABILITY

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

According to the present invention, there is no need to remove unaffected interspinous ligaments. Therefore, an easy surgical treatment is ensured that is less burdensome on the patient. In particular, during surgical treatment, resection and denervation of extensor muscles positioned at posterior laterals are not required.

Furthermore, according to the present invention, the narrowing of an intervertebral space can be prevented while maintaining a predetermined space between two adjacent spinous processes, and a relative displacement between a superior facet and an inferior facet does not occur.

In addition, an intervertebral implant of the present invention can be adapted to human spinous processes having different lower surface slopes.

### CONCLUSION

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. For example, one or more elements can be rearranged and/or combined, or additional elements may be added. Thus, the present invention is not intended to be limited to the embodiments shown herein but is to be accorded the widest scope consistent with the principles and novel features disclosed herein.

1. An intervertebral implant comprising a spacer having two opposing notches for receiving two adjacent spinous processes and a band securing the two spinous processes and the spacer,
the spacer comprising a through-hole bored through sides of the spacer to allow the band to pass therethrough and depressions curved inwardly from outsides of the spacer to facilitate fastening of the band passed through the through-hole, and
the band binding the two spinous processes and the spacer in a figure 8 form while passing through the through-hole to secure the two spinous processes and the spacer.

2. The intervertebral implant of claim 1, wherein the band secures the two spinous processes and the spacer by knotting both ends of the band with the figure 8 band portions.

3. The intervertebral implant of claim 2, wherein the band secures the two spinous processes and the spacer so that no relative displacement between an inferior facet of an upper spinous process of the two spinous processes and a superior facet of a lower spinous process of the two spinous processes occurs.

4. The intervertebral implant of claim 3, wherein the band secures the two spinous processes and the spacer so that lumbar lordosis is restored.

5. The intervertebral implant of claim 1, wherein at least one notch of the two opposing notches has a profile obtained by connecting a plurality of points by line segments of different functions.

6. The intervertebral implant of claim 5, wherein the plurality of points are connected by straight line segments with different slopes.

7. The intervertebral implant of claim 5, wherein the plurality of points are connected by curved line segments with predetermined curvatures.

8. The intervertebral implant of claim 5, wherein the profile of the at least one notch has a gently sloped shape from an insertion rear part to an insertion front part.

9. The intervertebral implant of claim 5, wherein when upper or lower surface slopes of spinous processes of a large number of individuals are assigned to n intervals, a representative value of a n-th interval is θₙ, the end of one of the two opposing notches near an insertion rear part is the origin in the x-y plane, the cumulative value of the slopes assigned to the n-th interval is Nₙ, and the plurality of the points are the origin and (Nₙ, Nₙ tan θₙ).

10. The intervertebral implant of claim 5, wherein when upper or lower surface slopes of spinous processes of a large number of individuals are assigned to n intervals, a representative value of a n-th interval is θₙ, the end of one of the two opposing notches near an insertion rear part is the origin in the x-y plane, the cumulative value of the slopes assigned to the n-th interval is Nₙ, a uniform function having a function value of 1 in the interval (Nₙ₋₁, Nₙ) is u(Nₙ₋₁, Nₙ), the number of assigned slopes is x, and a function y obtained by connecting the origin and (Nₙ, Nₙ tan θₙ) by straight line is given by:
$$y = {\sum }_{m = 1}^{n} \{ tan {θ}_{m} ( x - {N}_{m - 1} ) + tan {θ}_{m - 1} {N}_{m - 1} \} * u ( {N}_{m - 1} , {N}_{m} )$$
$${N}_{0} = 0 , {θ}_{0} = 0.$$

11. An intervertebral implant comprising:
a spacer having two opposing notches receiving two adjacent spinous processes;
an elastic folding portion connecting the two opposing notches and having an elastic restoring force against an external force generated from the two spinous processes;
two through-holes formed respectively on the two opposing notches; and
a band binding the spacer and the two spinous processes by passing through the two through-holes.

12. The intervertebral implant of claim 11, wherein the elastic folding portion comprises a first vertical straight line portion connected to one of the two opposing notches, a second vertical straight line portion connected to the other notch, and a curved portion connecting the two straight line portions.

13. The intervertebral implant of claim 11, wherein the two through-holes are separated from the elastic folding portion by a predetermined distance so that when the band passes through the two through-holes, the elastic folding portion is not contacted to the band.

14. The intervertebral implant of claim 11, wherein at least one notch of the two opposing notches has a profile obtained by connecting a plurality of points by line segments of different functions.

15. An intervertebra implant comprising:
an upper body having a first notch;
a lower body having a second notch opposite to the first notch;
a cylindrical receiver formed on a lower portion of the upper body; and
an insertion member being formed on an upper portion of the lower body, being partially inserted into the cylindrical receiver, and having a first angle portion formed near an insertion front part and a second angle portion formed near an insertion rear part, the first angle portion and the second angle portion having different slopes.

16. The intervertebral implant of claim 15, further comprising:
a band; and
band fixing projections formed respectively on the upper and lower bodies to bind the upper and lower bodies with the band.

17. The intervertebral implant of claim 16, wherein at least one of the first notch and the second notch has a profile obtained by connecting a plurality of points by line segments of different functions.

18. A method for determining a clustering of anatomical shapes using spinal images, the method comprising:
extracting a plurality of samples;
converting the samples to a digitized format;
assigning a first case to a first class;
image matching a subsequent case to a digitized representative image of the first class;
in the case of the subsequent case matches the digitized image of the first class within predetermined limits, correcting the first class in accordance with the image matching;
in the case of the subsequent case not matching the digitized image of the first class within the predetermined limits, establishing an additional class and assign the subsequent case to the additional class;
repeating the sequence of matching a subsequent case to the first class and each additional class;
in the case of no digitized images matching the digitized VOI of the n-th case, the n-th case is assigned to a newly created class P+1; and
selecting a minimum ratio for removal and removing cases with less than the selected minimum ratio as exceptional classes representative of exceptional and different shapes.

19. The method of claim 18, further comprising:
extracting, as the a plurality of samples, a plurality of volumes of interest (VOI) samples of spinal process shapes;
in the repeating of the sequence of matching a subsequent case to the first class and each additional class, upon comparing the digitized VOI of an n-th case with the digitized representative images of existing classes A through P, assigning the n-th case to its matched class;
in the case of no digitized images matching the digitized VOI of the n-th case, the n-th case is assigned to a newly created class P+1;
after removing exceptional classes, determine corrections of the representative image of each class, and in the case of correction, again perform the sequence of assigning a first case to a first class, image matching subsequent cases correcting the classes and establishing additional classes; and
calculating error rates of converged classes, and in the case of the number of classes less than a maximum number of permissible classes, iterating the sequence with an increased or decreased similarity threshold.
